SEQUENCE LISTING (2 sequences)

SEQ ID NO: 1 - DNA, 25 bp, Artificial
  Synthetic sequence
cctcttacctcagttacaatttata

SEQ ID NO: 2 - DNA, 18 bp, Artificial
  Synthetic sequence
gctattaccttaacccag

(19) United States
(12) Patent Application Publication
(10) Pub. No.: US 2010/0196277 A1
(43) Pub. Date: Aug. 5, 2010

(54) NANOPARTICLE COMPOSITIONS FOR CONTROLLED DELIVERY OF NUCLEIC ACIDS

(75) Inventors: Joseph M. DeSimone, Chapel Hill, NC (US); Andrew James Murphy, Chapel Hill, NC (US); Ashley Galloway, Cary, NC (US); Robby A. Petros, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The University of North Carolina at Chapel Hill, Chapel Hill, NC (US)

(21) Appl. No.: 12/444,662

(22) PCT Filed: Oct. 9, 2007
(86) PCT No.: PCT/US07/21680

§ 371 Date: Mar. 11, 2010

Related U.S. Application Data
(60) Provisional application No. 60/828,719, filed on Oct. 9, 2006.

(51) Int. Cl. A61K 49/00; A61K 9/00; A61K 31/7088; A61K 39/00

(52) U.S. Cl. 424/9.1; 424/484; 514/44 R; 514/44 A; 424/184.1

(57) ABSTRACT

Micro- and nano-particles are molded in micro- and nano-scale molds fabricated from non-wetting, low surface energy polymeric materials. The micro- and nano-particles can include pharmaceutical compositions, biologic drugs, drug compositions, organic materials, RNA, DNA, oligonucleotides, and the like.

### CROSS REFERENCE TO RELATED APPLICATIONS

The present application claims priority to U.S. Provisional Patent Application No. 60/828,719 filed Oct. 9, 2006, which is incorporated herein by reference in its entirety.

### GOVERNMENT INTEREST

A portion of the disclosure contained herein was made with U.S. Government support from the Science and Technology Center program of the National Science Foundation under Agreement No. CHE-9876674; the National Institutes of Health, Grant Nos. GM05299 and CA119343; from the Defense Advanced Research Projects Agency, Agreement No. W911NF-06-1-0343; and from the Juvenile Diabetes Research Fund, Agreement No.08-0477. The U.S. Government has certain rights to that portion of the disclosure.

### INCORPORATION BY REFERENCE

All documents referenced herein are hereby incorporated by reference as if set forth in their entirety herein, as well as all references cited therein.

### TECHNICAL FIELD

Generally, this invention relates to micro and/or nano scale particles provided as a delivery vehicle for nucleic acids. Prepared particles and methods of preparing the particles are disclosed, as well as methods of administrating the particles as therapeutics.

### ABBREVIATIONS

° C.=degrees Celsius

cm=centimeter

DMA=dimethylacrylate

DMPA=2,2-dimethoxy-2-phenylacetophenone

g=grams

h=hours

IL=imprint lithography

kg=kilograms

kPa=kilopascali

mL=milliliters

mm=millimeters

mmol=millimoles

m.p.=melting point

mW=milliwatts

NCM=nano-contact molding

NIL=nanoimprint lithography

nm=nanometers

PDMS=polydimethylsiloxane

PEG poly(ethylene glycol)

PFPE=perfluoropolyether

PLA poly(lactic acid)

SEM=scanning electron microscopy

Si=silicon

Tg=glass transition temperature

Tm=crystalline melting temperature

μm=micrometers

UV=ultraviolet

W=watts

### BACKGROUND

The transfer of genetic information into cells in the form of nucleic acids (“gene therapy”) is broadly appealing for therapeutic applications. The many recent developments in polymer science coupled with the difficulties in delivering labile nucleic acids as pharmaceutical agents have led to increased activity in this research area. A key challenge associated with gene therapy, whether ex vivo or in vivo, is the efficient transfer of the nucleic acid therapeutics into targeted cell populations. To be effective, the nucleic acids must not only enter the cell cytoplasm but must also do so in sufficient quantities to have the desired biological effect. Many approaches to increase the efficiency of gene transfer into cells complex the nucleic acid with delivery vectors that facilitate the transfer of the agent across the cell membrane into the cytoplasm and often into the nucleus. The ideal vector would protect the nucleic acid from degradation, facilitate cell membrane transfer by endocytosis or pinocytosis, and provide a mechanism for controllably releasing the nucleic acids once inside the cell. Such methods should be amenable for virtually any gene of interest and should permit the delivery of genetic material into a variety of cells and tissues, specific to the disease target and for reproducible and prolonged persistence over time to safely affect the therapeutic outcome. In addition, the ideal delivery vector must have a sufficient capacity to provide and protect required quantities of nucleic acid and also provide scaffolds on which targeting ligands may be attached to achieve site- or cell-specific delivery.

At present most commonly used techniques for nucleic acid delivery, such as microinjection, transfection using cationic liposomes, viral transfection or electroporation of oligonucleotide conjugates, have significant limitations. Methods such as microinjection or electroporation are simply not suitable for large-scale delivery of nucleic acids into living tissues in animals. Much work has been done with various types of polydisperse systems based on lipid or polymer particles for delivering therapeutic agents, such as micells, mixed micells, reversed micells, or unilamellar or multilamellar liposomes. For example, nucleic acid delivery has been demonstrated by the use of cationic liposomes such as LIPOFECTAMINE™, LIPOFECTIN™, CYTOFECTIN™, as well as transfection mediated by polymeric DNA-binding cations such as poly-L-lysine or polyethyleneimine. Direct in vivo gene transfer has recently been attempted with formulations of nucleic acid trapped in liposomes, as described in International patent publications WO03/059322A1; WO03/053409A1; WO00/03683A2; and U.S. Pat. No. 5,279,833, each of which is incorporated herein by reference in its entirety.

Liposome and other self assembled structures' use in therapy has gained only limited acceptance due to various problems such as instability of the carrier to be administered, leakage of the therapeutic agent from the systems, high costs of reagents, or poor storage stability. In general, such systems have proved to be inadequately effective in delivering efficacious amounts of the therapeutic agent to the target site. These methods can cause cytotoxicity and sensitivity to serum, antibiotics and certain cell culture media. The coupling of targeting ligands to the surface of liposomes presents a number of problems, mainly due to the fact that the liposomes have carbohydrate components and therefore contain multiple reactive groups. In addition, these methods are limited by low overall transfection efficiency and time-dependency. Liposomes, including polycationic liposomes, do not have the desirable sustained release properties that microspheres exhibit, as they tend to be less stable and to release their contents rapidly. Thus, for many purposes, liposomal delivery systems are not as effective as polymer particle delivery systems.

Viral vectors are regarded as the most efficient system, to date, for nucleic acid delivery and recombinant, replication-defective viral vectors have been used to transduce (via infection) cells, both ex vivo and in vivo. Such vectors have included retroviral, adenoviral and adeno-associated, and herpes viral vectors. While highly efficient at gene transfer, the major disadvantages associated with the use of viral vectors include the inability of many viral vectors to infect non-dividing cells; problems associated with insertional mutagenesis; inflammatory reactions to the virus and potential helper virus production; antibody responses to the viral coats; and the potential for production and transmission of harmful virus to other human patients.

Polymer microspheres and/or nanospheres may be used as vehicles for delivering drugs intracellularly, and for controlled sustained delivery for an extended period. Generally, microspheres and/or nanospheres comprise a biocompatible, biodegradable polymeric core having a bioactive agent incorporated therein. The methods of producing microspheres result in mostly spherical shapes with an average diameter of about 1 to 900 μm and considerable polydispersity. The same holds for nanospheres which are typically spherical and have an average diameter of less than 1 μm, usually less than about 300 nm, again with considerable polydispersity. From here forward in this Section, microspheres and nanospheres will both be referred to as “microspheres.” Advantages of polymer microsphere bioactive formulations include their ability to enter cells and penetrate intracellular junctions. Another advantage of microspheres is their ability to provide sustained or controlled release of bioactive agents. Thus, microspheres provide a means for controlled and/or sustained delivery of pharmaceutical and other bioactive agents to both intracellular as well as extracellular targets.

Due to their small size, polymeric microspheres have been found to evade recognition and uptake by the reticulo-endothelial system (RES), also referred to as the MPS system, and thus can circulate in the blood for an extended period, as described in Borchard, G. et al., Pharm Res. 7:1055-1058 (1996), which is incorporated herein by reference in its entirety. In addition, microspheres are able to extravasate at the pathological site, such as the leaky vasculature of a solid tumor, providing a passive targeting mechanism, as described in Yuan F. et al., Cancer Research 55:3752-3756 (1995); and Duncan, R. et al., STP Pharma. Sci. 4:237 (1996), each of which are incorporated herein by reference in their entirety. Active targeting can be accomplished with polymer particles through the use of surface functionalization. Functional groups allow the attachment of targeting molecules to the surface of the microspheres for enhanced site-specific delivery. Drug molecules or imaging agents can also be attached to the functionalized molecules on the surface of the microspheres directly or through the use of an appropriate linker. Further disclosure of such systems can be found in international patent publication WO 96/20698, which is incorporated herein by reference in its entirety.

One of the more popular methods is the use of biodegradable microspheres as a sustained release vehicle. The agents to be delivered are typically encapsulated in a polymer matrix which degrades over time, releasing the therapeutic, which are further disclosed in Langer, Science 1990, 249, 1527; WO002/32396; and U.S. Pat. No. 6,814,980, each of which are incorporated herein by reference in their entirety. Typical polymers used in preparing these particles are polyesters such as poly(glycolide-co-lactide) (PLGA), polyglycolic acid, and polyacrylic acid ester. These microspheres have the additional advantage of protecting the agent from degradation by the body. These particles, depending on their size, composition, and the agent being delivered, can be administered to an individual using any route available (e.g., oral, injection, inhalation administration). Other methods of making biodegradable microspheres include the use of a biodegradable cross-linker, as disclosed in International Patent Publication WO 2005/089106, which is incorporated herein by reference in its entirety. The rate of degradation can be controlled by changing the cross-linking ratio of a cross linker relative to the backbone monomer concentration. Varying this ratio can modulate the release rate of encapsulated drug. U.S. Pat. No. 6,521,431 describes several biodegradable cross-linkers that can be used in the preparation of biodegradable particles; the patent is hereby incorporated by reference in its entirety.

Despite the advantages of this delivery technique, efforts to formulate nucleic acids within microspheres have been hampered by several difficulties. For example, present production methods, such as spray-drying, emulsion, dispersion, and precipitation techniques, exhibit very low loading efficiencies, as most of the agent present in the formulation used to prepare the microspheres does not get incorporated into the microspheres. Methods that enhance the efficiency of nucleic acid incorporation would have the beneficial effect of requiring less therapeutic agent to produce an effective product, potentially providing both cost savings and lowered toxicological risk. Such methods might also increase the amount of nucleic acid incorporated into each particle, allowing the introduction of fewer particles into the treatment site to deliver a given amount of total nucleic acid to a patient. Moreover, incorporation of nucleic acid therapeutics into microspheres is plagued by fragmentation of the nucleic acid. In one common method, DNA microspheres are formed using a water-in-oil-in-water double emulsion method. Unfortunately, each of the two emulsifying steps frequently involves sonication, which causes fragmentation of the DNA. Additionally, these methods are limited to producing spherical shaped particles with inherent polydispersity. Filtration methods aid in reducing the polydispersity but add cost and complicate the fabrication procedures.

It is generally agreed that effective delivery of nucleic acid-based therapeutics is a major obstacle to their success in medicine, as disclosed in Henry, Chem. Eng. News. December 2003, 32-36, which is incorporated herein by reference in its entirety. Nucleic acid methodology has been extended to cultured mammalian cells, but its application in vivo in animals has been limited due to a lack of efficient delivery systems. Current polymer particle technologies have promise in many areas but clearly, improved methods of gene delivery are needed.

### SUMMARY

According to an embodiment of the present invention, a drug delivery vehicle includes a particle having a predetermined shape and a volume less than about 150 μm³ and an oligonucleotide coupled with the particle. In some embodiments, the concentration of the oligonucleotide is not in an equilibrium state in the particle. In other embodiments, the oligonucleotide includes between about 1 and about 75 weight percent of the particle. According to some embodiments, the volume of the particle is between about 5 μm³ and about 150 μm³. In some embodiments, the oligonucleotide comprises an RNA, siRNA, dsRNA, ssRNA, miRNA, rRNA, tRNA, snRNA, shRNA, DNA, ssDNA, dsDNA, plasmid DNA, or vaccine. According to some embodiments, the volume of the particle is not dependent on a size of the oligonucleotide, a concentration of the oligonucleotide, a charge of the oligonucleotide, charge density of the oligonucleotide, or chain length of the oligonucleotide. In other embodiments, the particle includes a poly(ethylene glycol). In some embodiments, the particle includes a disulfide bond. In yet other embodiments, the particle includes a biodegradable matrix. In some embodiments, the particle includes a matrix that is biodegradable in response to intracellular stimuli. According to some embodiments, the oligonucleotide is releasable coupled within the particle.

In some embodiments, a drug delivery vehicle includes a particle having a predetermined shape and a cross-sectional dimension of less than about 5 μm and an oligonucleotide coupled with the particle. In yet other embodiments, a drug delivery vehicle includes a particle having a predetermined shape and a volume less than about 150 μm³ and a vaccine coupled with the particle. In further embodiments, the present invention includes a drug delivery vehicle having a plurality of particles where each particle of the plurality of particles has a predetermined shape, each particle of the plurality of particles has a volume of less than about 150 μm³, and each particle of the plurality of particles has an oligonucleotide releasably coupled therewith.

In yet further embodiments of the present invention, a method for fabricating a drug delivery vehicle includes fabricating a particle, wherein the particle includes: poly(ethylene glycol), a polyacrylic acid ester, a disulfide bond, an oligonucleotide, a cross-sectional dimension less than about 5 μm, and a predetermined shape.

In other embodiments of the present invention, a method for fabricating a drug delivery vehicle includes; fabricating a mold from a non-wetting polymer, wherein the mold has a volume of less than about 150 μm³, introducing a biodegradable composition into the mold, introducing an oligonucleotide into the biodegradable composition, hardening the biodegradable composition and oligonucleotide in the mold such that a particle is formed in the mold, and extracting the particle from the mold.

### BRIEF DESCRIPTION OF THE DRAWINGS

Reference is made to the accompanying drawings in which are shown illustrative embodiments of the presently disclosed subject matter, from which its novel features and advantages will be apparent.

FIG. 1 shows an SEM micrograph of 2×2×1 μm positively charged DEDSMA particles according to an embodiment of the present invention.

FIG. 2 shows a fluorescent micrograph of 2×2×1 μm positively charged DEDSMA particles according to an embodiment of the present invention.

FIG. 3 shows a fluorescence micrograph of calcein cargo incorporated into 2 μm DEDSMA particles according to an embodiment of the present invention.

FIG. 4 shows 2×2×1 μm pDNA containing positively charged DEDSMA particles: Top Left: SEM, Top Right: DIC, Bottom Left: Particle-bound Polyflour 570 flourescence, Bottom Right: Fluorescein-labelled control plasmid fluorescence according to an embodiment of the present invention.

FIG. 5 shows 2×2×1 μm pDNA containing positively charged PEG particles: Top Left: SEM, Top Right: DIC, Bottom Left: Particle-bound Polyflour 570 flourescence, Bottom Right: Fluorescein-labelled control plasmid fluorescence according to an embodiment of the present invention.

FIGS. 6A and 6B show SEM images of particles containing fluorescently tagged mopholino antisense oligonucleotide cargo according to an embodiment of the present invention.

FIG. 7 shows RT-PCR data for particles containing mopholino antisense oligonucleotide cargo according to an embodiment of the present invention.

FIG. 8 shows percent splice shifting of particles containing mopholino antisense oligonucleotide cargo according to an embodiment of the present invention.

FIGS. 9A-9D show positively charged particles containing fluorescent oligonucleotide cargo. FIG. 9A is a DIC image, FIG. 9B is a fluorescent light microscopy image, and FIGS. 9C and 9D are SEM images according to an embodiment of the present invention.

FIGS. 10A-10B show SEM images of diethyldisulfide methacrylate and (2-acryloxyethyl) trimethyl ammonium chloride particles containing fluorescently tagged morpholino antisense oligonucleotide cargo according to an embodiment of the present invention.

FIGS. 11A-11B show SEM images of diethyldisulfide methacrylate, PEG monomethacrylate, and (2-acryloxyethyl)trimethyl ammonium chloride particles containing fluorescently tagged morpholino antisense oligonucleotide cargo according to an embodiment of the present invention.

FIGS. 12A-12B show SEM images of diethyldisulfide methacrylate and (2-acryloxyethyl)trimethyl ammonium chloride particles containing fluorescently tagged morpholino antisense oligonucleotide cargo according to an embodiment of the present invention.

FIGS. 13A-13B show SEM images of diethyldisulfide methacrylate and (2-acryloxyethyl)trimethyl ammonium chloride particles containing fluorescently tagged morpholino antisense oligonucleotide cargo according to an embodiment of the present invention.

FIGS. 14A-14B show SEM images of diethyldisulfide methacrylate and tertiary amine monomer particles containing fluorescently tagged morpholino antisense oligonucleotide cargo according to an embodiment of the present invention.

FIGS. 15A-15B show images of porous cationic PEG-diacrylate particles with plasmid DNA cargo. FIG. 15A is an optical image. FIG. 15B is an SEM image according to an embodiment of the present invention.

FIGS. 16A-16B show images of porous PEG-diacrylate particles with plasmid DNA cargo. FIG. 16A is an optical image. FIG. 16B is an SEM image according to an embodiment of the present invention.

FIGS. 17A-17B show images of porous cationic PEG-diacrylate particles with plasmid DNA cargo according to an embodiment of the present invention. FIG. 17A is an optical image. FIG. 17B is an SEM image.

FIG. 18 shows luminescence from transfected HeLa cells using PEG-diacrylate based PRINT particles containing pCMV Luciferase plasmid according to an embodiment of the present invention.

FIGS. 19A-19C show cationic disulfide particles containing ssDNA. FIG. 19A shows green fluorescence from FITC-tagged ssDNA according to an embodiment of the present invention. FIG. 19B shows Polyflour 570 red fluorescence from particles. FIG. 19C is an SEM image.

FIG. 20 is a chart of release of FITC-tagged ssDNA from cationic disulfide particles when in the presence of dithiothreitol (0.1 M in PBS, square data points) and in the absence of reductant (PBS, circular data points) according to an embodiment of the present invention.

FIGS. 21A-21D show SEM images of SEM images of 200 nm tall×200 nm diameter cylindrical streptavidin coated particles according to an embodiment of the present invention.

FIGS. 22A-22B show images of 200 nm tall×200 nm diameter cylindrical streptavidin (Alexa Fluor 488) coated particles. FIG. 22A is a DIC image and FIG. 22B is a fluorescence image according to an embodiment of the present invention.

FIG. 23A-23F show optical, fluorescent microscopy, and SEM images of PEG based particles containing fluorescently tagged anti-Luc siRNA according to an embodiment of the present invention.

FIGS. 24A-24B show images of PEG based particles containing fluorescently tagged anti-Luc siRNA, while the particles are in a mold. FIG. 24A is a fluorescence microscopy image and FIG. 24B is an optical microscopy image according to an embodiment of the present invention.

FIGS. 25A-25B show images of PEG based particles containing fluorescently tagged anti-Luc siRNA, after the particles have been harvested from the molds. FIG. 25A is a fluorescence microscopy image and FIG. 25B is an optical microscopy image according to an embodiment of the present invention.

FIG. 26 shows synthesis of an anisamide-based targeting ligand for incorporation into PRINT particles according to an embodiment of the present invention.

FIG. 27 shows an anisamide targeting ligand for PRINT particles according to an embodiment of the present invention.

FIGS. 28A-28B show SEM images of degradable particles with proton sponge monomer and fluorescently tagged anti-luciferase siRNA cargo according to an embodiment of the present invention.

FIG. 29 shows an SEM image of PEG based particles with proton sponge monomer and fluorescently tagged anti-luciferase siRNA cargo according to an embodiment of the present invention.

FIG. 30 is a plot of normalized mean fluorescence versus time for 30 wt % disulfide PRINT particles containing 2 wt % doxorubicin stirred in both PBS alone and 100 mM DTT in PBS according to an embodiment of the present invention.

FIG. 31 is a plot of cell viability as a function of particle dosing for HeLa cells (72 h dosing) according to an embodiment of the present invention.

FIG. 32 is a plot of antigen presentation for cells dosed with PRINT particles, supernatant from glutathione treatment, and free peptide according to an embodiment of the present invention.

### DETAILED DESCRIPTION

The present invention provides particle vectors, or vehicles, that carry and/or protect actives such as for example nucleic acids, facilitate entry into cells, and provide a mechanism for controllably releasing the active cargo. Such techniques are amenable to use with virtually any nucleic acid of interest and permit the introduction of genetic material into a variety of cells and tissues for consistent and prolonged dosing over time. In addition, the delivery particle vector is capable of holding a sufficient quantity of nucleic acids and acts as a scaffold to which ligands may be attached for preselected site or cell specific targeting.

According to some embodiments of the present invention the particle vectors include geometrically specific shaped particles fabricated from selectively biodegradable compositions that biodegrade upon exposure to intracellular stimulants. Methods of the present invention provide fabrication of the particle vectors into virtually any three dimensional shape with precise, repeatable, and highly controllable geometries. Moreover, methods of the present invention provide loading of the particle vectors with virtually any cargo, including in virtually any concentration, regardless of equilibrium states. The present invention thereby results in particle vectors loaded with a preselected concentration of a cargo, sized and shaped for cellular uptake, and constructed from controllable biodegradable compositions that, upon intracellular breakdown, release the cargo within the cell.

In some embodiments, the particles carry cargo including, but not limited to, biologically active cargo, an element, a molecule, a chemical substance, an agent, a therapeutic agent, a diagnostic agent, a pharmaceutical agent, a drug, a medication, genetic material, a nucleotide sequence, an amino-acid sequence, a ligand, an oligopeptide, a protein, a vaccine, a biologic, DNA, RNA, a cancer treatment, a viral treatment, a bacterial treatment, a fungal treatment, an auto-immune treatment, a psychotherapeutic agent, an imaging agent, a contrast agent, an antisense agent, radiotracers and/or radiopharmaceuticals combinations thereof, and the like.

In some embodiments, the particle vectors, or vehicles, of the present invention are fabricated in low surface energy polymeric molds. The molds, including fabrication of the molds, the materials from which the molds can be made, the general fabrication of particles in the molds, and release of particles from the molds are described in the applicants pending national and international patent applications incorporated herein by reference. For the sake of conciseness and simplicity, only a brief description of the molds, their fabrication, loading, particle formation, and release of particles therefrom will be described in the present application.

The particles described in some embodiments of the present invention can be utilized in applications, including, but not limited to, the delivery of a cargo.

### DEFINITIONS

“Adjuvant” means any compound which is a nonspecific modulator of the immune response. In certain embodiments, the adjuvant stimulates the immune response. Any adjuvant may be used in accordance with the present invention. A large number of adjuvant compounds is known; a useful compendium of many such compounds is prepared by the National Institutes of Health and can be found on the world wide web (http:/www.niaid.nih.govidaids/vaccine/pdf/compendium.pdf; see also Allison Dev. Biol. Stand. 92:3-11, 1998; Unkeless et al. Annu. Rev. Immunol. 6:251-281, 1998; and Phillips et al. Vaccine 10: 151-158, 1992, each of which is incorporated herein by reference.

“Animal” means humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Biodegradable” means compounds that are broken down or decomposed by natural biological processes. Biodegradable compounds, when introduced to a biologic fluid, are broken down by cellular machinery, proteins, enzymes, hydrolyzing chemicals, reducing agents, intracellular constituents, and the like into components that the cells can either reuse or dispose of without significant toxic effect on the cells (i.e., fewer than about 20% of the cells are killed). The term “biodegradable” as used herein refers to both enzymatic and non-enzymatic breakdown or degradation of the polymeric structure. Biodegradation can take place intracellularly or intercellularly. In certain preferred embodiments, the chemical reactions relied upon to break down the biodegradable compounds are uncatalyzed.

“Effective amount” means an amount necessary to elicit a desired biological response. As will be appreciated by those of ordinary skill in this art, the effective amount of particles may vary depending on such factors as the desired biological endpoint, the cargo to be delivered, the composition of the encapsulating matrix, the target tissue, etc. For example, the effective amount of particles containing a local anesthetic to be delivered to provide a nerve block is the amount that results in a reduction in sensation of a desired area for a desired length of time. In another example, the effective amount of particles containing an antigen to be delivered to immunize an individual is the amount that results in an immune response sufficient to prevent infection with an organism having the administered antigen.

“Polynucleotide” or “oligonucleotide” means a polymer of nucleotides. Typically, a polynucleotide comprises at least three nucleotides.

“RNA” means ribonucleic acid that synthesizes protein within a cell, transferring information from DNA to the protein-forming system of the cell. RNA is also involved in expression and repression of hereditary information and its four main types include heterogeneous nuclear RNA (hRNA); messenger RNA (mRNA), transfer RNA (tRNA), and ribosomal RNA (rRNA).

“microRNAs” (miRNAs) means small RNAs that play a broad role in eukaryotic gene expression. These small RNAs are used to block or destroy mRNAs with complementary sequences. This process is called RNAi (for RNA interference).

“mRNA” means messenger RNA and these represent the products of the majority of genes.

“rRNA” means ribosomal RNA and forms the structural component of the ribosome, the machine that translates mRNA into protein.

“tRNA” means types of RNA that form a “t” shape. Each of these

RNAs can recognize 1-3 codons (the 3 nucleotide code present in DNA and RNA) on one end via its anti-codon loop and is attached to an amino acid via its other end. As the ribosome “translates” mRNA into protein, tRNAs enter the ribosome and match amino acids to the mRNA's successive codons.

“snRNAs” mean small nuclear RNAs and are catalytic RNAs that perform mRNA splicing.

“Inhibit” or “down-regulate” means that the expression of a gene, level of RNAs or equivalent RNAs encoding one or more protein subunits, or activity of one or more protein subunits, such as pathogenic protein, viral protein or cancer related protein subunit(s), is reduced below that observed in the absence of the compounds or combination of compounds of the invention. In one embodiment, inhibition or down-regulation with an enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of viral or oncogenic RNA, protein, or protein subunits with a compound of the instant invention is greater in the presence of the compound than in its absence.

“Up-regulate” means that the expression of a gene, level of RNAs or equivalent RNAs encoding one or more protein subunits, or activity of one or more protein subunits, such as viral or oncogenic protein subunit(s), is greater than that observed in the absence of the compounds or combination of compounds of the invention. For example, the expression of a gene, such as a viral or cancer related gene, can be increased in order to treat, prevent, ameliorate, or modulate a pathological condition caused or exacerbated by an absence or low level of gene expression.

“Modulate” means that the expression of a gene, level of RNAs or equivalent RNAs encoding one or more protein subunits, or activity of one or more protein subunit(s) of a protein, for example a viral or cancer related protein is up-regulated or down-regulated, such that the expression, level, or activity is greater than or less than that observed in the absence of the compounds or combination of compounds of the invention.

“Nucleotide” means a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra) all of which are hereby incorporated by reference herein. There are several examples of modified nucleic acid bases known in the art as summarized by Limbach et al., 1994, Nucleic Acids Res. 22, 2183.

The presently disclosed subject matter will now be described more fully, beginning with an overview of fabrication of the low surface energy polymeric molds. The presently disclosed subject matter can, however, be embodied in different forms and should not be construed as limited to the embodiments set forth herein.

I. Particles

A. Fabrication of Particles Particles of some embodiments of the present invention are molded in low surface energy molds according to methods and materials described in the following patent applications: U.S. Provisional Patent Application Ser. No. 60/691,607, filed Jun. 17, 2005; U.S. Provisional Patent Application Ser. No. 60/714,961, filed Sep. 7, 2005; U.S. Provisional Patent Application Ser. No. 60/734,228, filed Nov. 7, 2005; U.S. Provisional Patent Application Ser. No. 60/762,802, filed Jan. 27, 2006; U.S. Provisional Patent Application Ser. No. 60/799,876 filed May 12, 2006; PCT International Application Serial No. PCT/US 06/23722, filed Jun. 19, 2006, entitled Nanoparticle Fabrication Methods, Systems and Materials; PCT International Application Serial No. PCT/US 06/34997, filed Sep. 7, 2006; U.S. Provisional Patent Application Ser. No. 60/798,858, filed May 9, 2006; U.S. Provisional Patent Application Ser. No. 60/800,478, filed May 15, 2006; U.S. Provisional Patent Application Ser. No. 60/811,136, filed Jun. 5, 2006; U.S. Provisional Patent Application Ser. No. 60/817,231, filed Jun. 27, 2006; U.S. Provisional Patent Application Ser. No. 60/831,372, filed Jul. 17, 2006; U.S. Provisional Patent Application Ser. No. 60/833,736, filed Jul. 27, 2006; U.S. Provisional Patent Application Ser. No. 60/841,581, filed Aug. 30, 2006; PCT International Patent Application Serial NO. PCT/US04/42706, filed Dec. 20, 2004, which is based on and claims priority to U.S. Provisional Patent Application Ser. No. 60/531,531, filed Dec. 19, 2003; U.S. Provisional Patent Application Ser. No. 60/583,170, filed Jun. 25, 2004; U.S. Provisional Patent Application Ser. No. 60/604,970, filed Aug. 27, 2004; U.S. Provisional Patent Application Ser. No. 60/531,531, filed Dec. 19, 2003, each of which is incorporated herein by reference in its entirety including all references cited therein.

In some embodiments, a particle of the present invention is fabricated in a non-wetting polymer mold. The mold may have cavities of a substantially predetermined shape, size, and/or arrangement. In some embodiments, the mold cavity has a volume of less than 150 μm³.

In some embodiments, a particle of the present invention is fabricated by introducing a composition disclosed herein into a mold cavity. In certain embodiments, the composition may contain cargo such as a biologically active cargo. In some embodiments, the composition in the cavity is treated to form a particle, whereby the particle mimics the size and/or shape of the mold cavity. The particles may then be extracted from the mold cavity.

The molds, materials, and methods are described in greater detail below and in the references incorporated herein.

In some embodiments, a non-wetting pattern replication method is used to generate isolated particles. In some embodiments, the isolated particles include isolated micro-particles. In some embodiments, the isolated particles include isolated nano-particles. In some embodiments, the isolated particles include a biodegradable material. In some embodiments, the isolated particles include a hydrophilic material. In some embodiments, the isolated particles include a hydrophobic material. In some embodiments, the isolated particles include a particular shape. In another embodiment, the isolated particles include or are configured to hold cargo. According to an alternative embodiment, the cargo protrudes from the surface of the isolated particle, thereby functionalizing the isolated particle. According to yet another embodiment, the cargo is completely contained within the isolated particle such that the cargo is stealthed or sheltered from an environment to which the isolated particle can be subjected. According to yet another embodiment, the cargo is contained substantially on the surface of the isolated particle. In a further embodiment, the cargo is associated with the isolated particle in a combination of one of the techniques herein, or the like.

According to another embodiment, the cargo is attached to the isolated particle by chemical binding or physical constraint. In some embodiments, the chemical binding includes, but is not limited to, covalent binding, ionic bonding, other intra- and inter-molecular forces, hydrogen bonding, van der Waals forces, combinations thereof, and the like. In some embodiments, the concentration of cargo associated with the particle is not limited by an equilibrium process, such that the amount of cargo in the particle can be rationally chosen.

In some embodiments, the particle fabrication methods further includes adding molecular modules, fragments, or domains to the composition to be molded. In some embodiments, the molecular modules, fragments, or domains impart functionality to the isolated particles. In some embodiments, the functionality imparted to the isolated particle includes a therapeutic functionality.

In some embodiments, a cargo such as a therapeutic agent, which may include a drug, a biologic, combinations thereof, and the like, is incorporated into the isolated particle. In some embodiments, the physiologically active drug is tethered to a linker to facilitate its incorporation into the isolated particle. In some embodiments, the domain of an enzyme or a catalyst is added to the isolated particle. In some embodiments, a ligand or an oligopeptide is added to the isolated particle. In some embodiments, the oligopeptide is functional. In some embodiments, the functional oligopeptide includes a cell targeting peptide. In some embodiments, the functional oligopeptide includes a cell penetrating peptide. In some embodiments an antibody or functional fragment thereof is added to the isolated particle.

In some embodiments, a binder is added to the isolated particle. In some embodiments, the shape of the isolated particle mimics a biological agent. In some embodiments, the method further includes a method for drug discovery.

B. Characteristics of Particles

A more detailed discussion of particular characteristics of the particles of the present invention follows. Generally, the particles can be fabricated from virtually any material composition; the particles can be fabricated in virtually any shape; the particles can include cargo such as drugs, therapeutic agents, biologic material, and the like; the particles can include diagnostic materials such as contrast agents and imaging agents; the particles can be functionalized to recognize particular cells, proteins, antigens, compositions, or molecules; combinations thereof; and the like.

i. Materials

In some embodiments, the material from which the particles are formed includes, without limitation, one or more of a polymer, a liquid polymer, a solution, a monomer, a plurality of monomers, a polymerization initiator, a polymerization catalyst, an inorganic precursor, an organic material, a natural product, a metal precursor, a pharmaceutical agent, a tag, a magnetic material, a paramagnetic material, a ligand, a cell penetrating peptide, a porogen, a surfactant, a plurality of immiscible liquids, a solvent, a charged species, combinations thereof, or the like.

In some embodiments, the monomer includes butadienes, styrenes, propene, acrylates, methacrylates, vinyl ketones, vinyl esters, vinyl acetates, vinyl chlorides, vinyl fluorides, vinyl ethers, acrylonitrile, methacrylnitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, epoxides, bisphenol A, alcohols, chlorosilanes, dihalides, dienes, alkyl olefins, ketones, aldehydes, vinylidene chloride, anhydrides, saccharide, acetylenes, naphthalenes, pyridines, lactams, lactones, acetals, thiiranes, episulfide, peptides, derivatives thereof, and combinations thereof.

In yet other embodiments, the polymer includes polyamides, proteins, polyesters, polystyrene, polyethers, polyketones, polysulfones, polyurethanes, polysiloxanes, polysilanes, cellulose, amylose, polyacetals, polyethylene, glycols, poly(acrylate)ₛ, poly(methacrylate)s, poly(vinyl alcohol), poly(vinylidene chloride), poly(vinyl acetate), poly(ethylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulfides, polyimides, liquid crystal polymers, heterocyclic polymers, polypeptides, conducting polymers including polyacetylene, polyquinoline, polyaniline, polypyrrole, polythiophene, and poly(p-phenylene), dendimers, fluoropolymers, derivatives thereof, combinations thereof, and the like.

In still further embodiments, the material from which the particles are formed includes a non-wetting agent. According to another embodiment, the material includes a liquid material in a single phase. In other embodiments, the liquid material includes a plurality of phases. In some embodiments, the liquid material includes, without limitation, one or more of multiple liquids, multiple immiscible liquids, surfactants, dispersions, emulsions, micro-emulsions, micelles, particulates, colloids, porogens, active ingredients, combinations thereof, or the like.

ii. Biodegradable/Time Release Particles

In some embodiments, particles are configured to controllably release cargo from the particle. In some embodiments, particles are fabricated from compositions that are designed to biodegrade over time. In some embodiments, the composition of the particle can be composed of a matrix that is biodegradable or bioresorbable such that after a predetermined time of exposure to known stimuli, the matrix of the particle will begin to break down and be absorbed. In some embodiments, the matrix of the resorbable particle can include a cargo such as a biologically active cargo. The cargo can be completely housed within the matrix of the particle such that the cargo is not recognized by an immune system, enzymes, or other conditions that will break down the cargo. In other embodiments, the cargo may be exposed or at the surface of the particle such that the particle has a functional quality. The matrix composition of particles of some embodiments of the present invention will now be described in greater detail.

In some embodiments, the polymeric microparticles and nanoparticles of the present invention can be prepared so as to be degradable, and suitably, to be biodegrading. The polymeric cross-linkers utilized in the present invention provide specific degradation points where breakdown of the polymeric cross-linker may occur. In some embodiments, these degradation points will be carboxylic acid ester groups or disulfide groups, though other biodegradable groups can be used in accordance with the present invention. In some embodiments, particles degrade upon crossing a cellular membrane into intracellular space. When the micro and nanoparticles of the present invention come in contact, for example, with proteins, enzymes, intracellular reductants, and/or hydrolyzing chemicals found in blood and other biological fluids, the polymeric cross-linkers are broken down. In some embodiments, this degradation creates linear polymeric end products that can be readily excreted from the body. The degradation also provides for a method via which encapsulated cargo, such as drugs or other agents can be released at a site within the body. In some embodiments, the rate of degradation and rate of release of cargo from the micro and nanoparticles can be controlled through the selection of a specific combination of polymeric backbones and cross-linkers with appropriate calibration of the ratio of cross-linker(s) to backbone polymer(s). Varying the amount of cross-linker(s) (e.g. 5%, 10%, 15%, 20%, 25%, or 30%) relative to backbone monomer(s) (i.e., the density of the cross-linker relative to the backbone monomers) allows for tailoring of the release rate of the encapsulated cargo.

A co-constituent of the particle, such as a polymer for example, can be cross-linked to varying degrees. Depending upon the amount of cross-linking of the polymer, another co-constituent of the particle, such as a cargo, can be configured to be released from the particle as desired. The cargo can be released with no restraint, controlled release, or can be completely restrained within the particle. In some embodiments, the particle can be functionalized, according to methods and materials disclosed herein, to target a specific biological site, cell, tissue, agent, combinations thereof, or the like. Upon interaction with the targeted biological stimulus, a co-constituent of the particle can be broken down to begin releasing the active co-constituent of the particle. In one example, the polymer can be poly(ethylene glycol) (PEG), which can be cross-linked between about 5% and about 100%. In one embodiment, when the PEG co-constituent is cross-linked about 100%, no cargo leaches out of the particle.

In certain embodiments, the particle includes a composition of material that imparts controlled, delayed, immediate, or sustained release of cargo of the particle or composition, such as for example, sustained drug release. According to some embodiments, materials and methods used to form controlled, delayed, immediate, or sustained release characteristics of the particles of the present invention include the materials, methods, and formulations disclosed in U.S. Patent Application Nos. 2006/0099262; 2006/0104909; 2006/0110462; 2006/0127484; 2004/0175428; 2004/0166157; and U.S. Pat. No. 6,964,780, each of which are incorporated herein by reference in their entirety.

In some embodiments, the particle includes a biodegradable polymer. In other embodiments, the polymer is modified to be a biodegradable polymer, e.g., a poly(ethylene glycol) that is functionalized with a disulfide group. In other embodiments, the polymer is modified to be a biodegradable polymer, e.g., a polyacrylic acid ester that is functionalized with a disulfide group. In some embodiments, the biodegradable polymer includes, without limitation, one or more of a polyester, a polyanhydride, a polyamide, a phosphorous-based polymer, a poly(cyanoacrylate), a polyurethane, a polyorthoester, a polydihydropyran, a polyacetal, combinations thereof, or the like. Further polymers that can be used in particles of the present invention are disclosed in Biodegradable Hydrogels for Drug Delivery, Park K., Shalaby W., Park H., CRC Press, 1993, which is incorporated herein by reference in its entirety.

In some embodiments, the polyester includes, without limitation, one or more of polylactic acid, polyglycolic acid, poly(hydroxybutyrate), poly(E-caprolactone), poly(β-malic acid), poly(dioxanones), combinations thereof, or the like. In some embodiments, the polyanhydride includes, without limitation, one or more of poly(sebacic acid), poly(adipic acid), poly(terpthalic acid), combinations thereof, or the like. In yet other embodiments, the polyamide includes, without limitation, one or more of poly(imino carbonates), polyaminoacids, combinations thereof, or the like.

According to some embodiments, the phosphorous-based polymer includes, without limitation, one or more of a polyphosphate, a polyphosphonate, a polyphosphazene, combinations thereof, or the like. Further, in some embodiments, the biodegradable polymer further includes a polymer that is responsive to a stimulus. In some embodiments, the stimulus includes, without limitation, one or more of pH, radiation, ionic strength, oxidation, reduction, temperature, an alternating magnetic field, an alternating electric field, combinations thereof, or the like. In some embodiments, the stimulus includes an alternating magnetic field. In some embodiments, a cargo such as a biologically active cargo can be combined with the particle material. In some embodiments, the cargo is a pharmaceutical agent. The pharmaceutical agent can be, but is not limited to, a drug, a peptide, RNA, RNAi, siRNA, shRNA, DNA, combinations thereof, or the like.

In some embodiments, the matrix composition of the particles is configured to biodegrade in the presence of an intercellular or intracellular stimulus. In some embodiments, the particles are configured to degrade in a reducing environment. In some embodiments, the particles contain crosslinking agents that are configured to degrade in the presence of an external stimulus. In some embodiments, the crosslinking agents are configured to degrade in the presence of a pH condition, a radiation condition, an ionic strength condition, an oxidation condition, a reduction condition, a temperature condition, an alternating magnetic field condition, an alternating electric field condition, combinations thereof, or the like. In some embodiments, the particles contain crosslinking agents that are configured to degrade in the presence of an external stimulus, a targeting ligand, and a therapeutic agent. In some embodiments, the therapeutic agent is a drug or a biologic. In some embodiments the therapeutic agent is DNA, RNA, shRNA, or siRNA.

In some embodiments, the particles are configured to degrade in the cytoplasm of a cell. In some embodiments, particles are configured to biodegrade in the cytoplasm of a cell and release a cargo such as a therapeutic agent. In some embodiments, the therapeutic agent is a drug or a biologic. In some embodiments the therapeutic agent is DNA, RNA, shRNA or siRNA. In some embodiments, the particles contain polyethylene glycol) and crosslinking agents that degrade in the presence of an external stimulus.

In some embodiments, the crosslinked polymer includes a hydrogel. In certain embodiments, the matrix is not degradable. The matrix may be configured to control diffusion of a cargo from the particle. In some embodiments, a particle is configured to release cargo without breaking chemical bonds of the composition from which the particle is formed. In some embodiments, a particle is configured to release cargo by passive release which may be related to swelling of the particle, diffusion of the cargo from the particle, pore size of the particle, cargo volume in relation to particle volume, or affinity of the cargo with the particle. In other embodiments, a particle is configured to release cargo by active release such as breakage of chemical bonds of the particle.

In some embodiments, the particle fabrication process provides, for example, control of particle matrix composition, the ability for the particle to carry a wide variety of cargos, the ability to functionalize the particle for targeting and enhanced circulation, and/or the versatility to configure the particle into different dosage forms, such as inhalation, dermatological, injectable, and oral, to name a few.

According to some embodiments, the matrix composition is tailored to provide control over biocompatibility. In some embodiments, the matrix composition is tailored to provide control over cargo release. The matrix composition, in some embodiments, contains biocompatible materials with solubility and/or philicity, controlled mesh density and charge, stimulated degradation, and/or shape and size specificity while maintaining relative monodispersity with respect to one, some, or all these traits within a given group of particles.

According to further embodiments, the method for making particles containing cargo does not require the cargo to be chemically modified. In one embodiment, the method for producing particles is a gentle processing technique that allows for high cargo loading without the need for covalent bonding. In one embodiment, cargo is physically entrapped within the particle due to interactions such as Van der Waals forces, electrostatic, hydrogen bonding, other intra- and inter-molecular forces, combinations thereof, and the like.

In some embodiments, the monomer, crosslinking agent, and photoinitiator used in fabricating the matrix composition of the invention are suitable for forming a hydrogel that is biocompatible and/or degradable. This degradation creates linear polymeric end products that can be readily excreted from a living the body, such as for example, a human, dog, cat, monkey, rat, mouse, horse, goat, rabbit, pig, cow, or the like. The degradation also provides for a method via which cargo, such as drugs or other agents can be released at a site within the body. In some embodiments, the rate of degradation and rate of release of cargo from the particles can be controlled through the selection of a specific combination of polymeric backbones and cross-linkers with appropriate calibration of the ratio of cross-linker(s) to backbone polymer(s). Varying the amount of cross-linker(s) (e.g. 5%, 10%, 15%, 20%, 25%, or 30%) relative to backbone monomer(s) (i.e., the density of the cross-linker relative to the backbone monomers) will allow for tailoring of the release rate of the encapsulated cargo.

Biodegradable network structures are prepared by placing covalent or non-covalent bonds within the network structure that are broken under biologically relevant conditions. In some embodiments, this involves the use of two separate structural motifs. In some embodiments, the biodegradable structure is either placed into (i) the polymer backbone or (ii) into the cross-linker structure.

In some embodiments, biodegradable polymers can be based on hydrophobic polymers like PLGA, poly(orthoesters), polyanhydrides, polyiminocarbonates, and others known to those skilled in the art which degrade hydrolytically into water-soluble monomers and oligomers. Other degradable polymers are based on naturally occurring polymers, e.g. polysaccharides or polypeptides. The degradation process is based on enzymatic hydrolysis of the polymer.

A further approach is to synthesize a polymer that contains an unstable crosslinker. In some embodiment, this crosslinker can degrade based through hydrolysis, enzymatic cleavage, changes in temperature, pH, or other environments such as oxidation or reduction. Crosslinking groups can include hydrolytically labile carbonate, ester, and phosphazene linkers, lactide or glycolide, and alpha hydroxy acids such as glycolic, succinic, or lactic acid. Cross-linkers of the present invention may also include a degradable region containing one or more groups such as anhydride, an orthoester, and/or a phosphoester. In certain cases the biodegradable region may contain at least one amide functionality. The cross-linker of the present invention may also include an ethylene glycol oligomer, oligo(ethylene glycol), poly(ethylene oxide), poly(vinyl pyrolidone), poly(propylene oxide), poly(ethyloxazoline), or combinations of these substances. In some embodiments, crosslinkers of the present invention include reduction/oxidation cleavable crosslinkers, such as a disulfide bridges, azo linkages, combinations thereof, or the like. Crosslinkers susceptible to pH changes are also included; these systems can be stable under acidic or basic conditions and start to degrade at blood pH or can be base- or acid-catalyzed.

Hydrolytically degradable crosslinking agents that may be used for forming degradable organic particles include, but are not limited to, poly(ε-caprolactone)-β-tetraethylene glycol-β-poly(ε-caprolactone)dimethacrylate, poly(ε-caprolactone)-b-poly(ethylene glycol)-β-poly(ε-caprolactone)dimethacrylate, poly(lactic acid)-β-tetraethylene glycol-β-poly(lactic acid)dimethacrylate, poly(lactic acid)-β-poly(ethylene glycol)-β-poly(lactic acid)dimethacrylate, poly(glycolic acid)-β-tetraethylene glycol-β-poly(glycolic acid)dimethacrylate, poly(glycolic acid)-β-poly(ethylene glycol)-β-poly(glycolic acid)dimethacrylate, poly(ε-caprolactone)-β-tetraethylene glycol-β-poly(ε-caprolactone)diacrylate, poly(ε-caprolactone)-β-poly(ethylene glycol)-β-poly(ε-caprolactone)diacrylate, poly(lactic acid)-β-tetraethylene glycol-β-poly(lactic acid)diacrylate, poly(lactic acid)-β-poly(ethylene glycol)-β-poly(lactic acid) diacrylate, poly(glycolic acid)-β-tetraethylene glycol-β-poly(glycolic acid)diacrylate, poly(glycolic acid)-β-poly(ethylene glycol)-β-poly(glycolic acid)diacrylate, and mixtures thereof. Further crosslinkers that can be used in particles of the present invention are disclosed in Biodegradable Hydrogels for Drug Delivery, Park K., Shalaby W., Park H., CRC Press, 1993, which is incorporated herein by reference in its entirety.

Enzymatically degradable crosslinking agents that may be used for forming degradable organic particle include, but are not limited to, crosslinking agents in which a short sequence of amino acids (for example, 3-5 amino acids) are linked to two methacrylate or acrylate groups. Examples of enzymatically degradable crosslinking agents include, but are not limited to, alanine-proline-glycine-leucine-poly(ethylene glycol)-alanine-proline-glycine-leucine)-diacrylate, alanine-proline-glycine-leucine-diacrylate, alanine-proline-glycine-leucine-poly(ethylene glycol)-alanine-proline-glycine-leucine)-dimethylacrylate-, and alanine-proline-glycine-leucine-dimethylacrylate, combinations thereof, and the like. Other enzymatically degradable crosslinking agents are disclosed in West & Hubbell (1999) Macromolecules 32(1):241-4, which is incorporated herein by reference in its entirety. Other enzymatically cleaved crosslinkers contain azobonds. In some embodiments a hydrolytically labile crosslinker can be fabricated for use in the particles of the present invention. An example of a hydrolytically labile crosslinker includes poly(εcaprolactone)-β-tetraethylene glycol-β-poly(εcapr-olactone)dimethacrylate.

In some embodiments, particles are fabricated from a matrix of polyethylene glycol) (PEG) diacrylate blended with (2-acryloxyethyl) trimethyl ammonium chloride (AETMAC). To this monomer blend 2,2′-diethoxyacetophenone photo-initiator can be added along with a biologically active cargo, such as an oligonucleotide. Particles, such as 200 nm tall by 200 nm diameter cylinders can be fabricated from the matrix and exposed to living cells. Once in the cytoplasm, the cargo (e.g., oligonucleotide) in the particles is released. In some embodiments, the particles break down in the cytoplasm. In some embodiments, the particles break down in response to predetermined stimuli in the cytoplasm. In some embodiments, the particles become hydrated and the cargo diffuses out. In some embodiments, after the cargo is freed into the cytoplasm from the particle the cargo treats the cell for a defective, diseased, or infected condition.

Disulfide crosslinkers have received much attention in designing drug delivery systems, due to their reversibility and relative stability in blood plasm. A review of such systems can be found by Saito, et al. Adv Drug Del Rev. 55 (2003) 199-215, which is incorporated herein by reference in its entirety. Preferrably, the disulfide crosslinker is water soluble. Examples include the following systems:

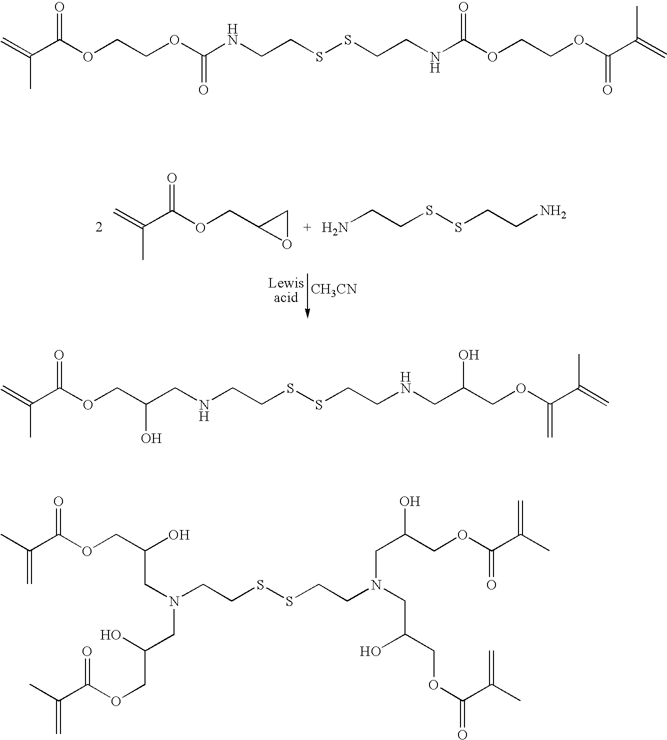

iii. Cargo

a. Particles Including Therapeutic Agents

In some embodiments, additional components are included with the material of the particle to functionalize the particle. According to these embodiments the additional components can be encased within the isolated particles, partially encased within the isolated particles, on the exterior surface of the isolated particles, combinations thereof, or the like. Additional components may be biologically active cargo, and can include, but are not limited to, drugs, biologics, oligonucleotides, polynucleotides, genetic material, one or more of a non-viral gene vector, DNA, RNA, RNAi, miRNA, mRNA, rRNA, tRNA, snRNA, shRNA, a viral particle, combinations thereof, and the like.

Derivatives of polynucleotides may also be used in some embodiments of the present invention. These derivatives can include modifications in the bases, sugars, and/or phosphate linkages of the polynucleotide. Modified bases include, but are not limited to, those found in the following nucleoside analogs: 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine. Modified sugars include, but are not limited to, 2′-fluororibose, ribose, 2′-deoxyribose, 3′-azido-2′, 3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphorarnidite linkages. Combinations of the various modifications may be used in a single polynucleotide. These modified polynucleotides can be provided by any means known in the art; however, as will be appreciated by those of skill in this art, the modified polynucleotides are preferably prepared using synthetic chemistry in vitro.

The polynucleotides to be delivered can be in any physical form, for example, the polynucleotide may be a circular plasmid, a linearized plasmid, a cosmid, a viral genome, a modified viral genome, an artificial chromosome, combinations thereof, or the like. The polynucleotide may be of any sequence. In certain embodiments, the polynucleotide encodes a protein or peptide. The encoded proteins may be enzymes, structural proteins, receptors, soluble receptors, ion channels, pharmaceutically active proteins, cytokines, interleukins, antibodies, antibody fragments, antigens, coagulation factors, albumin, growth factors, hormones, insulin, combinations thereof, and the like. The polynucleotide can also include regulatory regions to control the expression of a gene. These regulatory regions may include, but are not limited to, promoters, enhancer elements, repressor elements, TATA box, ribosomal binding sites, stop site for transcription, combinations thereof, and the like. In other embodiments, the polynucleotide is not intended to encode a protein, for example, the polynucleotide may be used to fix an error in the genome of the cell being transfected.

In some embodiments, the polynucleotide may also be provided as an antisense agent according to some embodiments. Antisense therapy is meant to include, e.g., administration or in situ provision of single- or double-stranded oligonucleotides or their derivatives which specifically hybridize, e.g., bind, under cellular conditions, with cellular mRNA and/or genomic DNA, or mutants thereof, so as to inhibit expression of the encoded protein, e.g., by inhibiting transcription and/or translation (Crooke, “Molecular mechanisms of action of antisense drugs” Biochim. Biophys. Acta 1489(1):31-44, 1999; Crooke, “Evaluating the mechanism of action of antiproliferative antisense drugs” Antisense Nucleic Acid Drug Dev. 10(2):123-126, discussion 127, 2000; Methods in Enzymology volumes 313-314, 1999; each of which is incorporated herein by reference). The binding can be by conventional base pair complementarity, or, for example, in the case of binding to DNA duplexes, through specific interactions in the major groove of the double helix (i.e., triple helix formation) (Chan et al., J. Mol. Med. 75(4):267-282, 1997; incorporated herein by reference).

In some embodiments, the polynucleotide includes RNA, a ribonucleic acid that synthesizes protein within a cell, transferring information from DNA to the protein-forming system of the cell; also involved in expression and repression of hereditary information; its four main types are: heterogeneous nuclear RNA (hRNA); messenger RNA (mRNA); transfer RNA (tRNA); and ribosomal RNA (rRNA).

In some embodiments, the polynucleotide includes microRNAs (miRNAs). MicroRNAs are small RNAs that play a broad role in eukaryotic gene expression. In some embodiments, the polynucleotide includes mRNA, or messenger RNA. These represent the products of the majority of genes, and these small RNAs are used to destroy mRNAs with complementary sequences. This process is called RNAi (for RNA interference). In some embodiments, the polynucleotide includes ribosomal RNA (rRNA). This forms the structural component of the ribosome, the machine that translates mRNA into protein. Interestingly, all the catalytic sites in the ribosome are formed by the bases coming off RNA. In some embodiments, the polynucleotide is tRNA. Each of these RNAs can recognize 1-3 codons (the 3 nucleotide code present in DNA and RNA) on one end via its anti-codon loop and is attached to an amino acid via its other end. As the ribosome “translates” mRNA into protein, tRNAs enter the ribosome and match amino acids to the mRNA's successive codons. In some embodiments, the polynucleotide includes small nuclear RNA. These are catalytic RNAs that perform mRNA splicing. In some embodiments, the polynucleotide includes miRNA, which stands for micro RNA. These small RNAs are used to block or destroy mRNAs with complementary sequences. This process is called RNAi (for RNA interference).

In some embodiments, the polynucleotide can be associated with other agents in the particles. Such agents can include poly-amines which neutralize the negative charge in the phosphate backbone of the polynucleotide. These agents may allow for the passage of the neutral complex through cellular and nuclear membranes. These agents can also protect the polynucleotide from degradation once the polynucleotide is in the cell.

In alternative embodiments, the polynucleotide to be delivered includes a sequence encoding an antigenic peptide or protein. Particles containing these polynucleotides can be delivered to a patient to induce an immunologic response sufficient to decrease the chance of a subsequent infection and/or lessen the symptoms associated with such an infection. The polynucleotide of these vaccines may be combined with interleukins, interferon, cytokines, and adjuvants such as cholera toxin, alum, Freund's adjuvant, combinations thereof, or the like. A large number of adjuvant compounds is known; a useful compendium of many such compounds is prepared by the National Institutes of Health and can be found on the world wide web (http:/www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10:151-158, 1992, each of which is incorporated herein by reference).

In some embodiments, polynucleotides to be delivered are used to modulate splicing of pre-mRNA. These splice switching oligonucleotides hybridize to splicing elements in pre-mRNA and redirect splicing from one splice variant to another. As a result the disease causing splice variant may be reduced and a therapeutic splice variant may be increased. The chemical composition of these polynucleotides may include modifications listed above or one or more of 2-deoxyribonucleotides, 2′O-methyl (2′-methoxy)ribonucleotides, 2′O-MOE (—O-ethyl-O-methyl)ribonucleotides, hexitol (HNA) nucleotides or nucleosides, 2′O-4′C-linked bicyclic ribofuranosyl (LNA) nucleotides or nucleosides, phosphorothioate analogs of any of the foregoing, methylphosphonate analogs of any of the foregoing, N3→P5′ phosphoramidate analogs of any of the foregoing and combinations thereof. Furthermore, the splice switching oligonucleotides (oligomers) may comprise of phosphorodiamidate morpholino nucleotide analogs and peptide nucleic acid (PNA) nucleotide analogs. Further detail is included in the following references which are incorporated in their entirety: U.S. Pat. No. 5,627,274; U.S. Pat. No. 5,665,593; U.S. Pat. No. 5,916,808; U.S. Pat. No. 5,976,879; Palma et al., “Efficient and persistent splice switching by systemically delivered LNA oligonucleotides in mice.” Mol. Ther. 2006 October; 14(4):471-5; Sazani P, Kole R., “Therapeutic potential of antisense oligonucleotides as modulators of alternative splicing.” J Clin Invest. 2003 August; 112(4):481-6; Sazani et al. “Systemically delivered antisense oligomers upregulate gene expression in mouse tissues.” Nat Biotechnol. 2002 December; 20(12):1228-33.

In some embodiments, the particle includes a therapeutic or diagnostic agent coupled with the particle. The therapeutic or diagnostic agent can be physically coupled or chemically coupled with the particle, encompassed within the particle, at least partially encompassed within the particle, coupled to the exterior of the particle, entangled within the matrix of the particle, crosslinked into the particle, covalently bonded to the matrix of the particle, held in the particle by hydrophobic/hydrophilic forces, combinations thereof, and the like. The therapeutic agent can be a drug, a biologic, a ligand, an oligopeptide, a cancer treating agent, a viral treating agent, a bacterial treating agent, a fungal treating agent, combinations thereof, or the like.

According to other embodiments, one or more other drugs can be included with the particles of the presently disclosed subject matter and can be found in Physicians' Desk Reference, Thomson Healthcare, 61^{st} ed. (2007), which is incorporated herein by reference in its entirety.

In some embodiments, the particle may be modified to include targeting agents since it is often desirable to target an active to a particular cell, collection of cells, or tissue. A variety of targeting agents that direct pharmaceutical compositions to particular cells are known in the art (see, for example, Cotten et al, Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, combinations thereof, or the like. The targeting agent can be used to target specific cells or tissues or can be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, combinations thereof, and the like. If the targeting agent is included throughout the particle, the targeting agent may be included in the mixture that used to form the particles. If the targeting agent is primarily on the surface of the particle, the targeting agent may be associated with (i.e., by covalent, hydrophobic, hydrogen boding, van der Waals, or other interactions) the formed particles using standard chemical techniques.

In some embodiments, one or more particles contain chemical moiety handles for the attachment of protein. In some embodiments, the protein is avidin. In some embodiments biotinylated reagents are subsequently bound to the avidin. In some embodiments the protein is a cell penetrating protein. In some embodiments, the protein is an antibody fragment. In one embodiment, the particles are used for specific targeting (e.g., breast tumors in female subjects). In some embodiments, the particles contain chemotherapeutics. In some embodiments, the particles are composed of a cross link density or mesh density designed to allow slow release of the chemotherapeutic. The term crosslink density means the mole fraction of prepolymer units that are crosslink points. Prepolymer units include monomers, macromonomers and the like.

In some embodiments, the physical properties of the particle are varied to enhance cellular uptake. In some embodiments, the size (e.g., mass, volume, length or other geometric dimension) of the particle is varied to enhance cellular uptake. In some embodiments, the charge of the particle is varied to enhance cellular uptake. In some embodiments, the charge of the particle ligand is varied to enhance cellular uptake. In some embodiments, the shape of the particle is varied to enhance cellular uptake.

b. Functionalization of Particles

In some embodiments, the particles are functionalized for targeting and enhanced circulation. In some embodiments, these features allow for tailored bioavailability. In one embodiment, the tailored bioavailability increases delivery effectiveness. In one embodiment, the tailored bioavailability reduces side effects. Particles may be functionalized according to methods and materials as described in the references incorporated herein, including U.S. Provisional Patent Application No. 60/841,581.

In some embodiments, the functionalized particles may include an agent selected from the group including dyes, fluorescent tags, radiolabeled tags, contrast agents, ligands, peptides, pharmaceutical agents, proteins, DNA, RNA, shRNA, siRNA, compounds and materials disclosed elsewhere herein, combinations thereof, and the like.

In some embodiments, the particles are configured to elicit an immune response. In some embodiments, the particles are configured to stimulate B-cells. In some embodiments, the B-cells are stimulated by targeting ligands covalently bound to the particles. In some embodiments, the B-cells are stimulated by haptens bound to the particles. In some embodiments, the B-cells are stimulated by antigens bound to the particles.

In some embodiments, the particles are functionalized with targeting ligands. In some embodiments, the particles are functionalized to target tumors. In some embodiments, the particles are functionalized to target breast tumors. In some embodiments, the particles are functionalized to target the HER2 receptor. In some embodiments, the particles are functionalized to target breast tumors and contain a chemotherapeutic. In some embodiments, the particles are functionalized to target dendritic cells.

c. Optimization of Particles

In some embodiments, fabrication of the particles of the present invention (PRINT or PRINT particles) and related techniques are combined with methods to control the location and orientation of chemical components within an individual object. In some embodiments, such methods improve the performance of an object by rationally structuring the object so that it is optimized for a particular application. In some embodiments, the method includes incorporating biological targeting agents into particles for drug delivery, vaccination, and other applications. In some embodiments, the method includes designing the particles to include a specific biological recognition motif. In some embodiments, the biological recognition motif includes biotin/avidin and/or other proteins.

In some embodiments, the method includes tailoring the chemical composition of these materials and controlling the reaction conditions, whereby it is then possible to organize the biorecognition motifs so that the efficacy of the particle is optimized. In some embodiments, the particles are designed and synthesized so that recognition elements are located on the surface of the particle in such a way to be accessible to cellular binding sites, wherein the core of the particle is preserved to contain bioactive agents, such as therapeutic molecules. In some embodiments, a non-wetting imprint lithography method is used to fabricate the objects, wherein the objects are optimized for a particular application by incorporating functional motifs, such as biorecognition agents, into the object composition. In some embodiments, the method further includes controlling the microscale and nanoscale structure of the object by using methods selected from the group including self-assembly, stepwise fabrication procedures, reaction conditions, chemical composition, crosslinking, branching, hydrogen bonding, ionic interactions, covalent interactions, and the like. In some embodiments, the method further includes controlling the microscale and nanoscale structure of the object by incorporating chemically organized precursors into the object. In some embodiments, the chemically organized precursors are selected from the group including block copolymers and core-shell structures.

In some embodiments, the physical properties of the particle are varied to enhance biodistribution. In some embodiments, the size (e.g., mass, volume, length or other geometric dimension) of the particle is varied to enhance biodistribution. In some embodiments, the charge of the particle matrix is varied to enhance biodistribution. In some embodiments, the charge of the particle ligand is varied to enhance biodistribution. In some embodiments, the shape of the particle is varied to enhance biodistribution. In some embodiments, the aspect ratio of the particles is varied to enhance biodistribution. According to some embodiments, the particle is hydrophilic such that the particle avoids clearance by biological organism, such as a human. In some embodiments, the type of targeting ligand is varied to enhance biodistribution. According to some embodiments, the particles have a predetermined zeta-potential.

In some embodiments, the physical properties of the particle are varied to enhance cellular response to the particles, such as adhesion, uptake, breakdown, release, combinations thereof, or the like. In some embodiments, the size (e.g., mass, volume, length or other geometric dimension) of the particle is varied to enhance cellular adhesion. In some embodiments, the charge of the particle matrix is varied to enhance cellular adhesion. In some embodiments, the chemistry of the ligand is varied to enhance cellular adhesion. In some embodiments, the charge of the particle ligand is varied to enhance cellular adhesion. In some embodiments, the shape of the particle is varied to enhance cellular adhesion.

According to some embodiments, material can be incorporated into a particle composition or as a particle according to the present invention, to treat or diagnose diseases including, but not limited to, allergies; anemia; anxiety disorders; autoimmune diseases; birth defects; blood disorders; bone diseases; cancers; circulation diseases; eye conditions; foodborne illnesses; gastrointestinal diseases; genetic disorders; heart diseases; hormonal disorders; impulse control disorders; infectious diseases; kidney diseases; leukodystrophies; liver diseases; mental health disorders; metabolic diseases; neurological disorders; pregnancy complications; prion diseases; prostate diseases; respiratory diseases; sexually transmitted diseases; skin conditions; thyroid diseases; vestibular disorders; waterborne illnesses; and other diseases such as diseases and conditions found at: http://www.mic.ki.se/Diseases/Alphalist.html, which is incorporated herein by reference in its entirety including each reference cited therein.

iv. Predetermined Particle Size and Shape

According to some embodiments, a particle has a substantially predetermined shape. According to some embodiments, a particle is formed that has a shape corresponding to a cavity of a mold (e.g., the particle has a shape reflecting the shape of the mold within which the particle was formed) of desired shape and is less than about 100 μm in a given dimension (e.g. minimum, intermediate, or maximum dimension). A particle may be measured in terms of a dimension of the particle. The dimension of the particle can be a predetermined dimension, a cross-sectional diameter, a circumferential dimension, or the like. The dimension can be measured across the largest portion of the particle that corresponds to the parameter being measured.

In one embodiment, the largest dimension of the particle is less than about 100 microns. In another embodiment, the largest dimension of the particle is less than about 90 microns. In another embodiment, the largest dimension of the particle is less than about 80 microns. In another embodiment, the largest dimension of the particle is less than about 70 microns. In another embodiment, the largest dimension of the particle is less than about 60 microns. In another embodiment, the largest dimension of the particle is less than about 50 microns. In another embodiment, the largest dimension of the particle is less than about 40 microns. In another embodiment, the largest dimension of the particle is less than about 30 microns. In another embodiment, the largest dimension of the particle is less than about 20 microns. In another embodiment, the largest dimension of the particle is less than about 10 microns. In another embodiment, the largest dimension of the particle is less than about 9 microns. In another embodiment, the largest dimension of the particle is less than about 8 microns. In another embodiment, the largest dimension of the particle is less than about 7 microns. In another embodiment, the largest dimension of the particle is less than about 6 microns. In another embodiment, the largest dimension of the particle is less than about 5 microns. In another embodiment, the largest dimension of the particle is less than about 4 microns. In another embodiment, the largest dimension of the particle is less than about 3 microns. In another embodiment, the largest dimension of the particle is less than about 2 microns. In another embodiment, the largest dimension of the particle is less than about 1 microns.

In another embodiment, the largest dimension of the particle is less than about 950 nanometers. In another embodiment, the largest dimension of the particle is less than about 900 nanometers. In another embodiment, the largest dimension of the particle is less than about 850 nanometers. In another embodiment, the largest dimension of the particle is less than about 800 nanometers. In another embodiment, the largest dimension of the particle is less than about 750 nanometers. In another embodiment, the largest dimension of the particle is less than about 700 nanometers. In another embodiment, the largest dimension of the particle is less than about 650 nanometers. In another embodiment, the largest dimension of the particle is less than about 600 nanometers. In another embodiment, the largest dimension of the particle is less than about 550 nanometers. In another embodiment, the largest dimension of the particle is less than about 500 nanometers. In another embodiment, the largest dimension of the particle is less than about 450 nanometers. In another embodiment, the largest dimension of the particle is less than about 400 nanometers. In another embodiment, the largest dimension of the particle is less than about 350 nanometers. In another embodiment, the largest dimension of the particle is less than about 300 nanometers. In another embodiment, the largest dimension of the particle is less than about 250 nanometers. In another embodiment, the largest dimension of the particle is less than about 200 nanometers. In another embodiment, the largest dimension of the particle is less than about 150 nanometers. In another embodiment, the largest dimension of the particle is less than about 100 nanometers. In another embodiment, the largest dimension of the particle is less than about 50 nanometers. In another embodiment, the largest dimension of the particle is less than about 45 nanometers. In another embodiment, the largest dimension of the particle is less than about 40 nanometers. In another embodiment, the largest dimension of the particle is less than about 35 nanometers. In another embodiment, the largest dimension of the particle is less than about 30 nanometers. In another embodiment, the largest dimension of the particle is less than about 25 nanometers. In another embodiment, the largest dimension of the particle is less than about 20 nanometers. In another embodiment, the largest dimension of the particle is less than about 15 nanometers. In another embodiment, the largest dimension of the particle is less than about 10 nanometers. In another embodiment, the largest dimension of the particle is less than about 9 nanometers. In another embodiment, the largest dimension of the particle is less than about 8 nanometers. In another embodiment, the largest dimension of the particle is less than about 7 nanometers. In another embodiment, the largest dimension of the particle is less than about 6 nanometers. In another embodiment, the largest dimension of the particle is less than about 5 nanometers. In another embodiment, the largest dimension of the particle is less than about 4 nanometers. In another embodiment, the largest dimension of the particle is less than about 3 nanometers. In another embodiment, the largest dimension of the particle is less than about 2 nanometers. In another embodiment, the largest dimension of the particle is less than about 1 nanometer.

The particle can be of an organic material or an inorganic material and can be one uniform compound or component or a mixture of compounds or components. In some embodiments, an organic material molded with the materials and methods of the present invention includes a material that includes a carbon molecule. According to some embodiments, the particle can be of a high molecular weight material. According to some embodiments, a particle is composed of a matrix that has a predetermined surface energy. In some embodiments, the material that forms the particle includes more than about 50 percent liquid. In some embodiments, the material that forms the particle includes less than about 50 percent liquid. In some embodiments, the material that forms the particle includes less than about 10 percent liquid.

According to some embodiments, a plurality of particles may have a substantially equivalent substantially predetermined size and/or shape. According to other embodiments, the particles produced by the methods and materials of the presently disclosed subject matter have a poly dispersion index (i.e., normalized size distribution) of between about 0.80 and about 1.20, between about 0.90 and about 1.10, between about 0.95 and about 1.05, between about 0.99 and about 1.01, between about 0.999 and about 1.001, combinations thereof, and the like. Furthermore, in other embodiments the particle has a mono-dispersity. According to some embodiments, dispersity is calculated by averaging a dimension of the particles. In some embodiments, the dispersity is based on, for example, surface area, length, width, height, mass, volume, porosity, combinations thereof, and the like.

According to other embodiments, particles of many predetermined regular and irregular shape and size configurations can be made with the materials and methods of the presently disclosed subject matter. Examples of representative particle shapes that can be made using the materials and methods of the presently disclosed subject matter include, but are not limited to, non-spherical, spherical, viral shaped, bacteria shaped, cell shaped, rod shaped, chiral shaped, right triangle shaped, flat shaped (e.g., with a thickness of about 2 nm, disc shaped with a thickness of greater than about 2 nm, or the like), boomerang shaped, combinations thereof, and the like.

In some embodiments, a non-spherical particle has a surface area that is greater than the surface area of spherical particle of the same volume. In some embodiments, the number of surface ligands on the particle is greater than the number of surface ligands on a spherical particle of the same volume.

In some embodiments, the particles are shaped to mimic natural structures. In some embodiments, the particles are substantially cell-shaped. In some embodiments, the particles are substantially red blood cell-shaped. In some embodiments, the particles are substantially red blood cell-shaped and composed of a matrix with a modulus less than 1 MPa. In some embodiments, the particles are shaped to mimic natural structures and contain a therapeutic agent, a contrast agent, a targeting ligand, combination thereof, and the like.

In some embodiments, the volume of a particle corresponds to a mold cavity. In some embodiments, the volume of a particle is not dependent on the size of cargo in the particle, the concentration of cargo in the particle, the charge of cargo in the particle, the charge density of cargo in the particle, or the chain length of an oligonucleotide cargo in the particle. In some embodiments, the cargo constitutes less than 75 weight percent of the particle.

### II. Uses of Particles

In some embodiments, a method of delivering cargo such as a therapeutic agent to a target is disclosed, the method including: providing a particle produced as described herein; admixing the therapeutic agent with the particle; and delivering the particle including the therapeutic agent to the target. In some embodiments, a particle is used as a therapeutic agent delivery vehicle.

In some embodiments, the therapeutic agent includes a drug. In some embodiments, the therapeutic agent includes genetic material. In some embodiments, the genetic material includes, without limitation, one or more of a non-viral gene vector, DNA, RNA, RNAi, a viral particle, combinations thereof, or the like.

In some embodiments, a particle may deliver a cargo such as a biologically active cargo to treat a disease. In some embodiments, a particle is delivered to a desired location in a patient. In some embodiments, the particle is delivered to a patient where the particle crosses a cellular membrane into intracellular space and releases the cargo to treat a disease. The particle may release the cargo actively or passively.

In some embodiments, the particle has a diameter of less than 100 microns. In some embodiments, the particle has a diameter of less than 10 microns. In some embodiments, the particle has a diameter of less than 1 micron. In some embodiments, the particle has a diameter of less than 100 nm. In some embodiments, the particle has a diameter of less than 10 nm.

In some embodiments, the particle includes a crosslinked polymer. In some embodiments, the particle includes a biodegradable polymer. In some embodiments, a biodegradable polymer can be a polymer that undergoes a reduction in molecular weight upon either a change in biological condition or exposure to a biological agent. In some embodiments, the biodegradable polymer includes, without limitation, one or more of a polyester, a polyanhydride, a polyamide, a phosphorous-based polymer, a poly(cyanoacrylate), a polyurethane, a polyorthoester, a polydihydropyran, a polyacetal, combinations thereof, or the like. In some embodiments, the polymer is modified to be a biodegradable polymer (e.g. a poly(ethylene glycol) that is functionalized with a disulfide group). In some embodiments, the polyester includes, without limitation, one or more of polylactic acid, polyglycolic acid, poly(hydroxybutyrate), poly(ε-caprolactone), poly(β-malic acid), poly(dioxanones), combinations thereof, or the like. In some embodiments, the polyanhydride includes, without limitation, one or more of poly(sebacic acid), poly(adipic acid), poly(terpthalic acid), combinations thereof, or the like. In some embodiments, the polyamide includes, without limitation, one or more of a poly(imino carbonate), a polyaminoacid, combinations thereof, or the like. In some embodiments, the phosphorous-based polymer includes, without limitation, one or more of polyphosphates, polyphosphonates, polyphosphazenes, combinations thereof, or the like. In some embodiments, the polymer is responsive to stimuli, such as pH, radiation, oxidation, reduction, ionic strength, temperature, alternating magnetic or electric fields, acoustic forces, ultrasonic forces, time, combinations thereof, and the like.

Responses to such stimuli can include swelling, bond cleavage, heating, combinations thereof, or the like, which can facilitate release of the isolated particle's cargo, degradation of the isolated particle itself, combinations thereof, and the like.

In some embodiments, the particle includes a hydrogel. In certain embodiments, the hydrogel is not degradable. The particle may be configured to control diffusion of a cargo from the particle. In some embodiments, a particle releases cargo without breaking chemical bonds of the particle. In some embodiments, a particle releases cargo by passive release which may be related to swelling of the particle, diffusion of the cargo from the particle, pore size of the particle, cargo volume in relation to particle volume, or affinity of the cargo with the particle. In other embodiments, a particle releases cargo by active release such as breakage of chemical bonds of the particle.

In some embodiments, the target includes, without limitation, one or more of a cell-targeting peptide, a cell-penetrating peptide, an integrin receptor peptide (GRGDSP), a melanocyte stimulating hormone, a vasoactive intestional peptide, an anti-Her2 mouse antibody, a vitamin, combinations thereof, or the like.

In one embodiment, the presently disclosed subject matter provides a method for modifying a particle surface. In one embodiment the method of modifying a particle surface includes: (a) providing particles in or on at least one of: (i) a patterned template; or (ii) a substrate; (b) disposing a solution containing a modifying group in or on at least one of: (i) the patterned template; or (ii) the substrate; and (c) removing excess unreacted modifying groups.

In one embodiment of the method for modifying a particle, the modifying group chemically attaches to the particle through a linking group. In another embodiment of the method for modifying a particle, the linker group includes, without limitation, one or more of sulfides, amines, carboxylic acids, acid chlorides, alcohols, alkenes, alkyl halides, isocyanates, combinations thereof, or the like. In another embodiment, the method of modifying the particles includes a modifying agent that includes, without limitation, one or more of dyes, fluorescence tags, radiolabeled tags, contrast agents, ligands, peptides, antibodies or fragments thereof, pharmaceutical agents, proteins, DNA, RNA, siRNA, shRNA, combinations thereof, or the like.

With respect to the methods of the presently disclosed subject matter, an animal subject can be treated. The term “subject” as used herein refers to a vertebrate species. The methods of the presently claimed subject matter are particularly useful in the diagnosis of warm-blooded vertebrates. Thus, the presently claimed subject matter concerns mammals. In some embodiments provided is the diagnosis and/or treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the diagnosis and/or treatment of livestock, including, but not limited to domesticated swine (pigs and hogs), ruminants, horses, poultry, and the like.

The following references are incorporated herein by reference in their entirety. Published International PCT Application No. WO2004081666 to DeSimone et al., U.S. Pat. No. 6,528,080 to Dunn et al.; U.S. Pat. No. 6,592,579 to Arndt et al., Published International PCT Application No. WO0066192 to Jordan; Hilger, I. et al., Radiology 570-575 (2001); Mornet, S. et al., J. Mat. Chem., 2161-2175 (2004); Berry, C. C. et al., J. Phys. D: Applied Physics 36, R198-R206 (2003); Babincova, M. et al., Bioelectrochemistry 55, 17-19 (2002); Wolf, S. A. et al., Science 16, 1488-1495 (2001); and Sun, S. et al., Science 287, 1989-1992 (2000); U.S. Pat. No. 6,159,443 to Hallahan; and Published PCT Application No. WO 03/066066 to Hallahan et al.

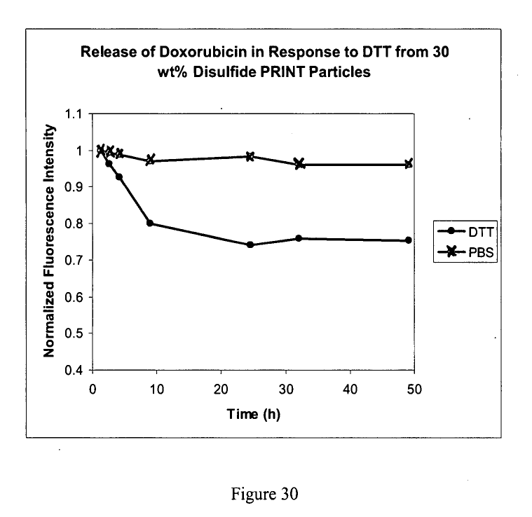

In some embodiments, the particles of the present invention include doxorubicin as the cargo. In some embodiments, particles with doxorubicin as cargo include disulfide based 2×2×2 micrometer avidin surface-functionalized particles. In some embodiments the disulfide crosslinker consists of about 30 wt % of the particle matrix materials. According to FIG. 30, doxorubicin loaded disulfide based 2×2×2 micrometer avidin surface-functionalized particles, containing about 30 wt % disulfide crosslinker show varying doxorubicin release times. As shown in FIG. 30, as represented by release of fluorophore/doxorubicin over time, the aliquot with a reducing agent, such as dithiothreitol, broke down and released more rapidly than the aliquot in a buffered saline, such as phosphate buffered saline alone.

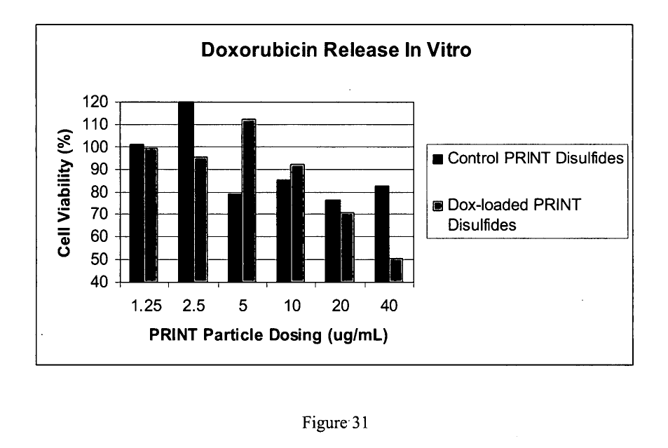

According to some embodiments, cell viability can be dependent on particle dosing, as shown in FIG. 31. In some embodiments, cells dosed with 40 micrograms per milliliter of 30 wt % disulfide crosslinked particles consisting of 2 wt % doxorubicin had about fifty percent less viability as cells dosed at 1.25 micrograms per milliliter with the same particle composition.

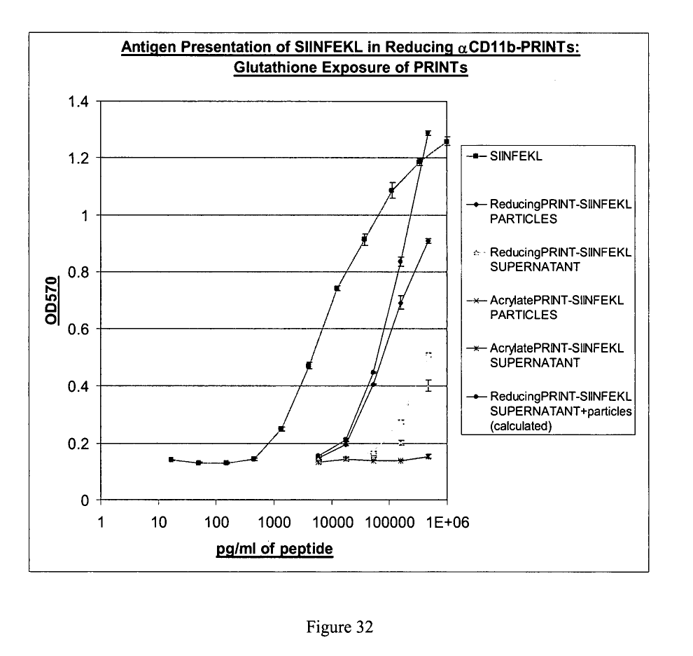

In another embodiment, degradable disulfide based particles of the present invention controllably degrade to release a cargo. As shown in FIG. 32, degradable disulfide based particles of the present invention are more efficient than non-degradable diacrylate based particles at stimulating targeted cells, thereby implying that a cargo was released from the disulfide based particles upon particle degradation and not passively diffused from the non-degradable diacrylate based particle.

In some embodiments, the particles of the present invention are functionalized with a ligand binding site. Particle matrix materials can include a functional group, such as an amine which can be biotinylated with biotin which can be subsequently bound to an avidin to provide cell specific targeted particles. According to an embodiment of the present invention, a particle can be formed from a matrix including 2-aminoethyl-methacrylate. After particle formation, as disclosed herein, in some embodiments the particle can be treated with biotin and acetic anhydride to biotinylate the biotin to the amine functional group of 2-aminoethyl-methacrylate. Following biotinylation of the particles of the present invention, avidin can be bound with the biotin of the biotinylated particles by mixing the biotinylated particles with avidin. Because avidin has four binding sites, each avidin molecule bound to the biotinylated particle has between 1 and 3 binding sites unoccupied and available for binding ligands.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist. Furthermore, it should be appreciated that all pharmaceutical compositions included in this specification shall include all pharmaceutically acceptable salts thereof.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Encapsulated DNA in 200 nm×200 nm×1 μm Bar-Shaped Poly(Lactic Acid) Particles

A patterned perfluoropolyether (PFPE) mold is generated by pouring a PFPE-dimethacrylate (PFPE-DMA) containing 1-hydroxycyclohexyl phenyl ketone over a silicon substrate patterned with 200 nm×200 nm×1 μm bar shapes. The substrate is then subjected to UV light (λ=365 nm) for 10 minutes while under a nitrogen purge. The fully cured PFPE-DMA mold is then released from the silicon master. Flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl)silane via vapor deposition in a desiccator for 20 minutes. Separately, a solution of 0.01 wt % 24 base pair DNA and 5 wt % poly(lactic acid) in ethanol is formulated. 200 μL of this ethanol solution is then placed on the treated silicon wafer and the patterned PFPE mold placed on top of it. The substrate is then placed in a molding apparatus and a small pressure is applied to push out excess PEG/FDG solution. The small pressure should be at least about 100 N/cm². The entire apparatus is then placed under vacuum for 2 hours. DNA-containing poly(lactic acid) particles will be observed after separation of the PFPE mold and the treated silicon wafer using optical microscopy.

### Example 2

### Synthesis of Degradable Crosslinkers for Hydrolysable PRINT Particles

Bis(ethylene methacrylate)disulfide (DEDSMA) was synthesized using methods described in Li et al. Macromolecules 2005, 38, 8155-8162 from 2-hydroxyethane disulfide and methacroyl chloride (Scheme 1). Analogously, bis(8-hydroxy-3,6-dioxaoctyl methacrylate) disulfide (TEDSMA) was synthesized from bis(8-hydroxy-3,6-dioxaoctyl)disulfide (Lang et al. Langmuir 1994, 10, 197-210). Methacroyl chloride (0.834 g, 8 mmole) was slowly added to a stirred solution of bis(8-hydroxy-3,6-dioxaoctyl)disulfide (0.662 g, 2 mmole) and triethylamine (2 mL) in acetonitrile (30 mL) chilled in an ice bath. The reaction was allowed to warm to room temperature and stirred for 16 hours. The mixture was diluted with 5% NaOH solution (50 mL) and stirred for an additional hour. The mixture was extracted with 2×60 mL of methylene chloride, the organic layer was washed 3×100 mL of 1 M NaOH, dried with anhydrous K₂CO₂, and filtered. Removal of the solvent yielded 0.860 g of the TEDSMA as a pale yellow oil. ¹H NMR (CDCl₃) δ=6.11 (2H, s), 5.55 (2H, s), 4.29 (4H, t), 3.51-3.8 (16H, m), 2.85 (4H, t), 1.93 (6H, s).

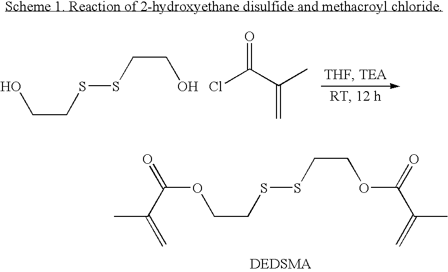

### Example 3

### Fabrication of 2 μm Positively Charged DEDSMA Particles

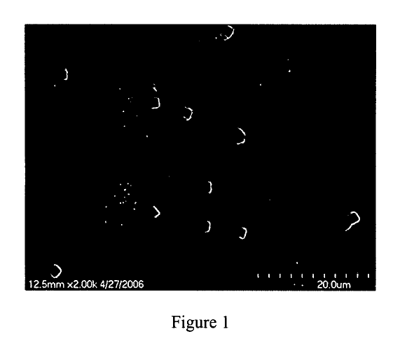

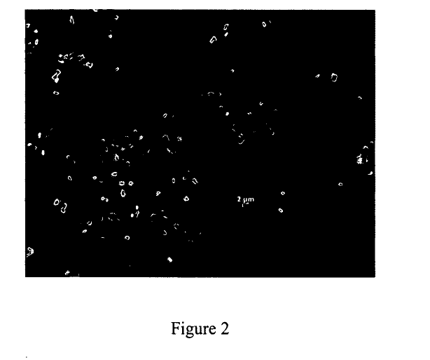

A patterned perfluoropolyether (PFPE) mold was generated by pouring a PFPE-dimethacrylate (PFPE-DMA) containing 1-hydroxycyclohexyl phenyl ketone over a silicon substrate patterned with 2 μm rectangles. A poly(dimethylsiloxane) mold was used to confine the liquid PFPE-DMA to the desired area. The apparatus was then subjected to UV light (λ=365 nm) for 10 minutes while under a nitrogen purge. The fully cured PFPE-DMA mold was then released from the silicon master. Separately, a mixture composed of acryloxyethyltrimethylammonium chloride (24.4 mg), DEDSMA (213.0 mg), Polyfluor 570 (2.5 mg), 2,2′ diethoxyacetophenone (5.0 mg), methanol (39.0 mg), acetonitrile (39.0 mg), water (8.0 mg), and N,N-dimethylformamide (6.6 mg) was prepared. This mixture was spotted directly onto the patterned PFPE-DMA surface and covered with a separated unpatterned PFPE-DMA surface. The mold and surface were placed in molding apparatus, purge with N₂ for ten minutes, and placed under at least 500 N/cm² pressure for 2 hours. The entire apparatus was then subjected to UV light (λ=365 nm) for 40 minutes while maintaining nitrogen purge. DEDSMA particles were harvested on glass slide using cyanoacrylate adhesive. The particles were purified by dissolving the adhesive layer with acetone followed by centrifugation of the suspended particles (see FIGS. 1 and 2).

### Example 4

### Encapsulation of Calcein Inside 2 μm Positively Charged DEDSMA Particles

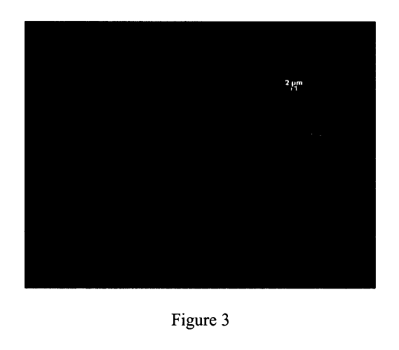

A patterned perfluoropolyether (PFPE) mold was generated by pouring a PFPE-dimethacrylate (PFPE-DMA) containing 1-hydroxycyclohexyl phenyl ketone over a silicon substrate patterned with 2 μm rectangles. A poly(dimethylsiloxane) mold was used to confine the liquid PFPE-DMA to the desired area. The apparatus was then subjected to UV light (λ=365 nm) for 10 minutes while under a nitrogen purge. The fully cured PFPE-DMA mold was then released from the silicon master. Separately, a mixture composed of acryloxyethyltrimethylammonium chloride (3.4 mg), DEDSMA (29.7 mg), calcein (0.7 mg), Polyfluor 570 (0.35 mg), diethoxyacetophenone (0.7 mg), methanol (5.45 mg), acetonitrile (5.45 mg), water (1.11 mg), and N,N-dimethylformamide (6.6 mg) was prepared. This mixture was spotted directly onto the patterned PFPE-DMA surface and covered with a separated unpatterned PFPE-DMA surface. The mold and surface were placed in molding apparatus, purge with N₂ for ten minutes, and placed under at least 500 N/cm² pressure for 2 hours. The entire apparatus was then subjected to UV light (λ=365 nm) for 40 minutes while maintaining nitrogen purge. Calcein containing DEDSMA particles were harvested on glass slide using cyanoacrylate adhesive. The particles were purified by dissolving the adhesive layer with acetone followed by centrifugation of the suspended particles (see FIG. 3).

### Example 5

### Encapsulation of Plasmid DNA into Charged DEDSMA Particles

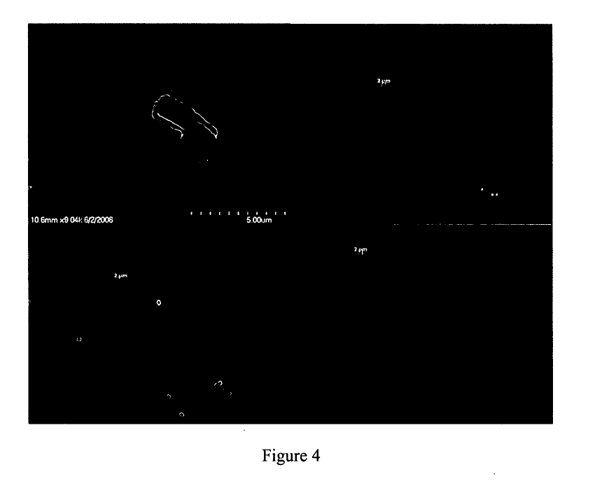

A patterned perfluoropolyether (PFPE) mold was generated by pouring a PFPE-dimethacrylate (PFPE-DMA) containing 1-hydroxycyclohexyl phenyl ketone over a silicon substrate patterned with 2 μm rectangles. A poly(dimethylsiloxane) mold was used to confine the liquid PFPE-DMA to the desired area. The apparatus was then subjected to UV light (λ=365 nm) for 10 minutes while under a nitrogen purge. The fully cured PFPE-DMA mold was then released from the silicon master. Separately, 0.5 μg of fluorescein-labelled plasmid DNA (Mirus Biotech) as a 0.25 μg/μL solution in TE buffer and a 2.0 μg of pSV β-galactosidase control vector (Promega) as a 1.0 μg/μL solution in TE buffer were sequentially added to a mixture composed of acryloxyethyltrimethylammonium chloride (1.44 mg), DEDSMA (12.7 mg), Polyfluor 570 (Polysciences, 0.08 mg), 1-hydroxycyclohexyl phenyl ketone (0.28 mg), methanol (5.96 mg), acetonitrile (5.96 mg), water (0.64 mg), and N,N-dimethylformamide (14.16 mg). This mixture was spotted directly onto the patterned PFPE-DMA surface and covered with a separated unpatterned PFPE-DMA surface. The mold and surface were placed in molding apparatus, purge with N₂ for ten minutes, and placed under at least 500 N/cm² pressure for 2 hours. The entire apparatus was then subjected to UV light (λ=365 nm) for 40 minutes while maintaining nitrogen purge. These particles were harvested on glass slide using cyanoacrylate adhesive. The particles were purified by dissolving the adhesive layer with acetone followed by centrifugation of the suspended particles (see FIG. 4).

### Example 6

### Encapsulation of Plasmid DNA into PEG Particles

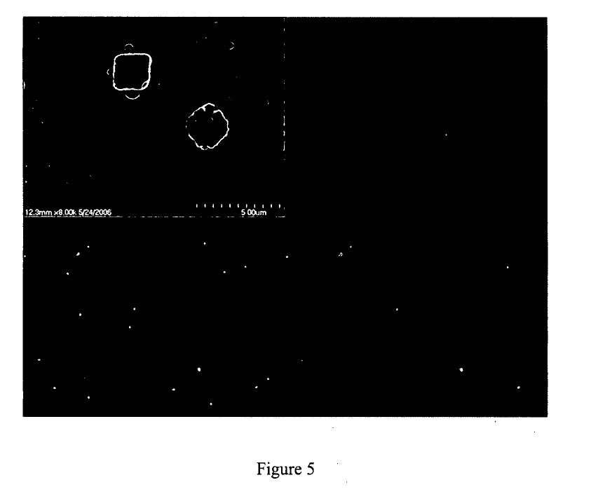

A patterned perfluoropolyether (PFPE) mold was generated by pouring a PFPE-dimethacrylate (PFPE-DMA) containing 1-hydroxycyclohexyl phenyl ketone over a silicon substrate patterned with 2 μm rectangles. A poly(dimethylsiloxane) mold was used to confine the liquid PFPE-DMA to the desired area. The apparatus was then subjected to UV light (λ=365 nm) for 10 minutes while under a nitrogen purge. The fully cured PFPE-DMA mold was then released from the silicon master. Separately, 0.5 μg of fluorescein-labelled plasmid DNA (Mirus Biotech) as a 0.25 μg/μL solution in TE buffer and a 2.0 μg of pSV μ-galactosidase control vector (Promega) as a 1.0 μg/μL solution in TE buffer were sequentially added to a mixture composed of acryloxyethyltrimethylammonium chloride (1.2 mg), polyethylene glycol diacrylate (n=9) (10.56 mg), Polyfluor 570 (Polysciences, 0.12 mg), diethoxyacetophenone (0.12 mg), methanol (1.5 mg), water (0.31 mg), and N,N-dimethylformamide (7.2 mg). This mixture was spotted directly onto the patterned PFPE-DMA surface and covered with a separated unpatterned PFPE-DMA surface. The mold and surface were placed in molding apparatus, purge with N₂ for ten minutes, and placed under at least 500 N/cm² pressure for 2 hours. The entire apparatus was then subjected to UV light (λ=365 nm) for 40 minutes while maintaining nitrogen purge. These particles were harvested on glass slide using cyanoacrylate adhesive. The particles were purified by dissolving the adhesive layer with acetone followed by centrifugation of the suspended particles (see FIG. 5).

The following references may provide information and techniques to supplement some of the techniques and parameters of the present examples, therefore, the references are incorporated by reference herein in their entirety including any and all references cited therein. Li, Y., and Armes, S. P. Synthesis and Chemical Degradation of Branched Vinyl Polymers Prepared via ATRP: Use of a Cleavable Disulfide-Based Branching Agent. Macromolecules 2005; 38: 8155-8162; and Lang, H., Duschl, C., and Vogel, H. (1994), A new class of thiolipids for the attachment of lipid bilayers on gold surfaces. Langmuir 10, 197-210.

### Example 7

### Cellular Uptake of DEDSMA PRINT Particles

The DEDSMA particles fabricated using PRINT were dispersed in 250 μL of water to be used in cellular uptake experiments. These particles were exposed to NIH 3T3 (mouse embryonic) cells at a final concentration of particles of 60 μg/mL. The particles and cells were incubated for 4 hrs at 5% CO₂ at 37° C. The cells were then characterized via confocal microscopy.

### Example 8

### Fabrication of a Degradable Crosslinker

Hydrolytically labile crosslinker, poly(.epsilon.-caprolactone)-b-tetraethylene glycol-b-poly(.epsilon.-caprolactone)dimethacrylate was synthesized according to previously described techniques (Sawhney et al. (1993) Macromolecules 26(4): 581-587). Briefly, tetraethylene glycol (5 ml) was reacted with .epsilon.-caprolactone (15-25 ml) at 140.degree C. in the presence of stannous octoate for 6 hours under vacuum. The reaction was cooled to room temperature and diluted with methylene chloride (50 ml). Triethylamine was added to the reaction mixture in 1.25 molar excess. Methacryloyl chloride (1.25 molar excess) was added dropwise to the reaction mixture. The reaction was continued at 4 degree C. under nitrogen overnight and at room temperature for 24 hours. The final product, poly(.epsilon.-caprolactone)-b-tetraethylene glycol-b-poly(.epsilon.-caprolactone)dimethacrylate, was verified with ¹H-NMR.

### Example 9

### Fabrication of PEG Based 200 nm Diameter Cylinders (200 nm Height) with Fluorescently Tagged Morpholino Antisense Oligonucleotide Cargo

An 8 inch master silicon substrate patterned with 200 nm tall×200 nm diameter cylindrical shapes was placed under a UV source. A patterned perfluoropolyether (PFPE) mold was generated by pouring 20 mL of PFPE-dimethacrylate (PFPE-DMA) synthesized by methods found in PCT US04/42706, containing 2,2-diethoxyacetophenone onto the patterned silicon substrate. The UV source containing the silicon substrate covered with PFPE-DMA was flushed with nitrogen for 2 minutes to remove oxygen. The apparatus was then subjected to UV light (λ=365 nm) for 3 minutes. The fully cured PFPE-DMA mold is then released from the silicon master.

Separately, a poly(ethylene glycol) (PEG) diacrylate (n=9) (88.84%) was blended with (2-acryloxyethyl)trimethyl ammonium chloride (AETMAC) (9.8%). To this monomer blend was added 1.02% by weight 2,2 diethoxyacetophenone photo-initiator and 0.34 wt % modified antisense oligonucleotide with the sequence CCTCTTACCTCAGTTACAATTTATA (SEQ ID NO: 1) containing a morpholino backbone and a 3′ fluorescein label. The solution was then diluted with 1:1 water/DMF to enhance miscibility resulting in a 94% solids solution. Following this, 15 μL of the above monomer blend was evenly spotted onto the PFPE-DMA mold. A polyethylene film measuring 8×10 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber is purged with Nitrogen for 2 minutes and then exposed to UV light (λ=365 nm) at greater than 20 mW/cm² for 3 minutes.

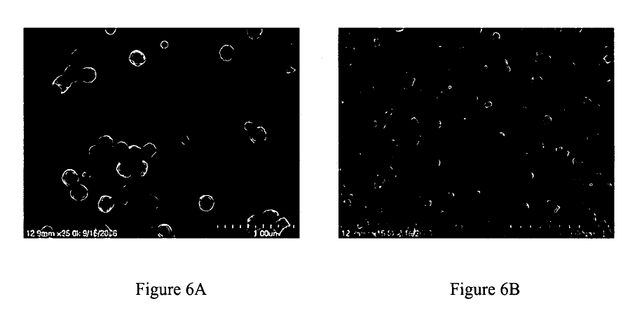

The cured mold is then removed from the UV chamber. 300 μL of doubly distilled filtered (0.22 μm) water is placed atop the mold. A glass slide is used to scrape the water across the mold and release the particles into the water, turning the water cloudy. The scraping of the mold continues for several passes until no more particles are released. The cloudy water suspension is then collected and filtered thru a 20 μm filter. The filtrate is then collected and concentrated using a 0.1 μm centricon tube. Finally, the particles are resuspended from the filter into pure water. SEM images of the oligo containing particles are shown in FIGS. 6A and 6B.

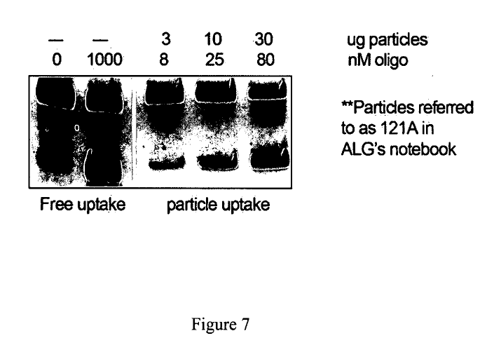

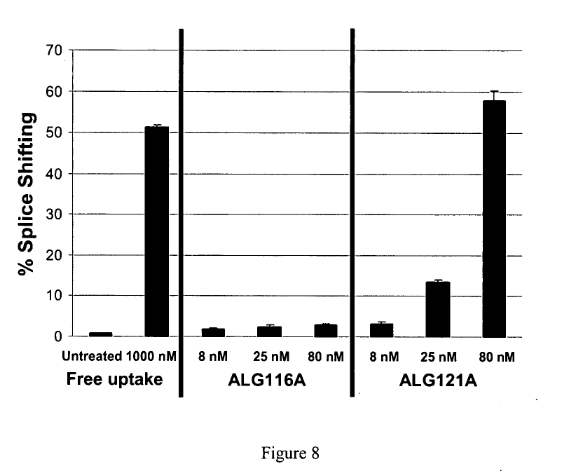

Next, these particles were incubated for 48 hours with HeLa cells possessing a mutation at intron 2 of the β-globin gene that causes an aberrant splicing of the pre-mRNA leading to a β-globin deficiency. The encapsulated cargo of the particles, a morpholino antisense oligonucleotide, is known to correct this aberrant splicing, thus restoring correct splicing of the pre-mRNA. In this experiment, the HeLa cells were incubated with particles for 48 hours. After 48 hours, the cells were lysed and the mRNA was isolated. RT-PCR was run to determine the effect of the particles on the mRNA production in these cells. Referring to FIG. 7, the data suggest a nice dose dependence restoration of mRNA, as shown in FIG. 8. When quantified, the PEG particles containing only 80 nm of oligo (121A) show a greater degree of splice shifting than the free oligonucleotide at greater than 10× concentration in vitro. Moreover, the splice switching levels that are produced at 80 nM is roughly the same order of magnitude as produced with lipofectamine.

### Example 10

### Fabrication of Degradable 2×2×1 μm Boxes with Fluorescently Tagged Morpholino Antisense Oligonucleotide Cargo

A 6 inch diameter circular silicon substrate patterned with 2×2×1 μm rectangular shapes is encased in an airtight UV-transparent mold maker. A patterned perfluoropolyether (PFPE) mold is generated by adding 10 mL of PFPE-dimethacrylate (PFPE-DMA) containing 2,2-diethoxyacetophenone into the mold maker in between the patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA mold is then released from the silicon master in the mold maker. Similarly, a flat, uniform, non-wetting surface is generated by encasing a blank silicon wafer into the airtight UV-transparent surface maker. The non-patterned perfluoropolyether (PFPE) surface is generated by adding 10 mL of PFPE-dimethacrylate (PFPE-DMA) containing 2,2-diethoxyacetophenone into the surface maker in between the non-patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA surface is then released from the silicon surface in the surface maker.

Separately, a diethyldisulfide methacrylate (82.63%) is blended with amino ethyl trimethylammonium chloride (AETMAC) (9.06%). To this monomer blend was added 7.77% by weight hydroxyl cyclohexyl phenyl ketone (HCPK) photo initiator, 0.5% rhodamine, and 0.03 wt % modified antisense oligonucleotide with the sequence CCTCTTACCTCAGTTACAATTTATA (SEQ ID NO: 1) containing a morpholino backbone and a 3′ fluorescein label. The solution was then diluted with 2.5:2.5:1 methanol:acetonitrile:water to enhance miscibility resulting a 58% solids solution.

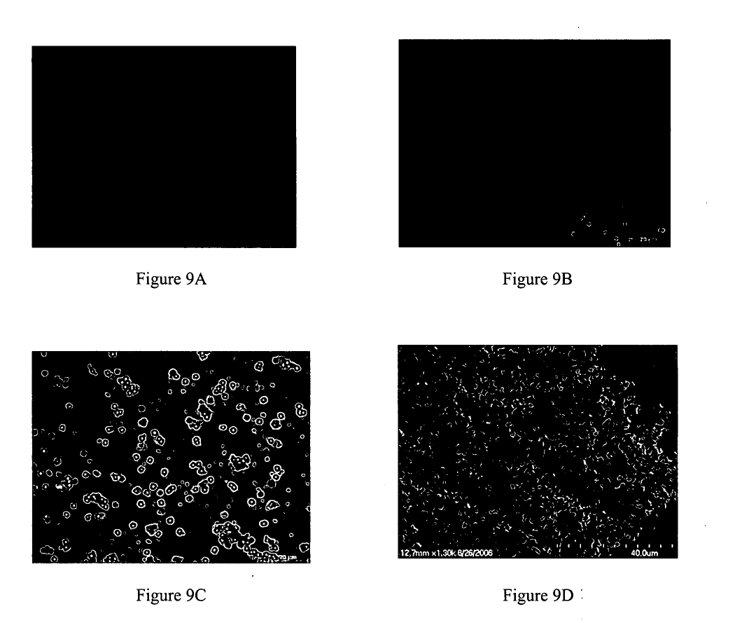

Following this, 0.1 mL of the above monomer blend is evenly spotted onto the flat PFPE-DMA surface and then the patterned PFPE-DMA mold placed on top of it. Pressure is applied with a roller for a few strokes to help spread the monomer solution. The surface and mold are then placed atop a PDMS dome under a UV light with an attached pressure clamp (particle maker). Once inside the particle maker, the apparatus is purged with nitrogen for 6 minutes at 50 kPa. A pressure of 1 ton is applied to the mold and surface to remove any excess monomer solution. At this point, nitrogen flow is shut off. After 1 hour of pressing, the entire apparatus is subjected to UV light (λ=365 nm) for 45 minutes. After curing, the mold and surface are separated to reveal discrete 2×2×1 μm oligonucleotide containing particles in the mold observable by light microscopy. The harvesting process begins by dispersing a thin layer of cyanoacrylate monomer onto the PFPE-DMA mold filled with particles. The PFPE-DMA mold is immediately placed onto a glass slide and the cyanoacrylate is allowed to polymerize in an anionic fashion for one minute. The mold is removed and the particles are embedded in the soluble adhesive layer, which provides isolated, harvested colloidal particle dispersions upon dissolution of the soluble adhesive polymer layer in acetone. Particles embedded in the harvesting layer, or dispersed in acetone can be visualized by light microscopy or SEM. The fluorescently labeled oligonucleotide cargo can be visualized using a fluorescent lamp attached to the light microscope. The dissolved poly(cyanoacrylate) can remain with the particles in solution, or can be removed via centrifugation. As shown in FIGS. 9A-9D, the harvested 2×2××1 μm positively charged particles contain the fluorescent oligonucleotide condensed inside. The particles are imaged by DIC, fluorescent light microscopy, and SEM.

### Example 11

### Fabrication of Degradable 200 nm Diameter Cylinders (200 nm Height) with Fluorescently Tagged Morpholino Antisense Oligonucleotide Cargo

An 8 inch silicon substrate patterned with 200 nm tall×200 nm diameter cylindrical shapes is placed under a UV source. A patterned perfluoropolyether (PFPE) mold is generated by pouring 20 mL of PFPE-dimethacrylate (PFPE-DMA) containing 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

Separately, a degradable monomer diethyldisulfide methacrylate (87.415%) is blended with (2-acryloxyethyl)trimethyl ammonium chloride (AETMAC) (10.001%). To this monomer blend was added 2.580% by weight hydroxyl cyclohexyl phenyl ketone (HCPK) photo initiator and 0.004 wt % modified antisense oligonucleotide with the sequence CCTCTTACCTCAGTTACAATTTATA (SEQ ID NO: 1) containing a morpholino backbone and a 3′ fluorescein label. The solution was then diluted with DMSO to enhance miscibility resulting in a 60% solids solution. Following this, 15 μL of the above monomer blend is evenly spotted onto the PFPE-DMA mold. A chlorotrifluoroethylene film measuring 8.5×11 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber is purged with Nitrogen for 2 minutes and then exposed to UV light at greater than 20 mW/cm² for 3 minutes.

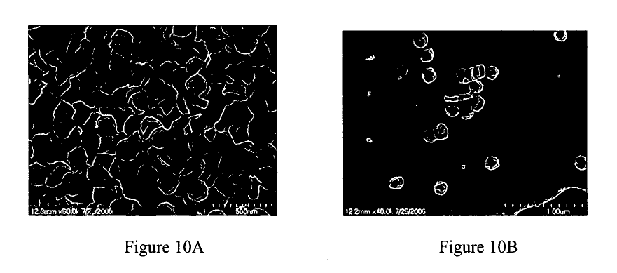

The cured mold is then removed from the UV chamber. 300 μL of doubly distilled filtered (0.22 μm) water is placed atop the mold. A glass slide is used to scrape the water across the mold and release the particles into the water, turning the water cloudy. The scraping of the mold continues for several passes until no more particles are released. The cloudy water suspension is then collected and filtered thru a 20 μm filter. The filtrate is then collected and concentrated using a 0.1 μm centricon tube. Finally, the particles are resuspended from the filter into pure water. FIGS. 10A and 10B show SEM images of the oligo containing particles.

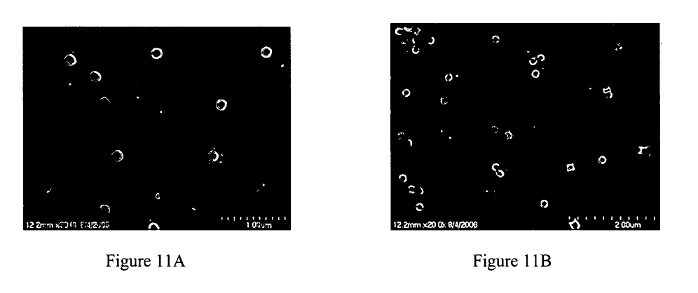

A second particle formulation was also fabricated in the same manner. This particle combination contained both PEG monomethacrylate and the degradable monomer. The degradable monomer, diethyldisulfide methacrylate (45.13%) was blended is blended with both PEG monomethacrylate (43.12%) and (2-acryloxyethyl)trimethyl ammonium chloride (AETMAC) (9.72%). To this monomer blend was added 2.02% by weight 2,2′ diethoxyacetophenone photo-initiator and 0.01 wt % modified antisense oligonucleotide with the sequence CCTCTTACCTCAGTTACAATTTATA (SEQ ID NO: 1) containing a morpholino backbone and a 3′ fluorescein label. The solution was then diluted with 20:1 DMSO:H₂O to enhance miscibility resulting a 73% solids solution. The particles are then fabricated in the same manner as the other degradable particles as described above. FIGS. 11A and 11B show SEM images of the oligo containing particles.

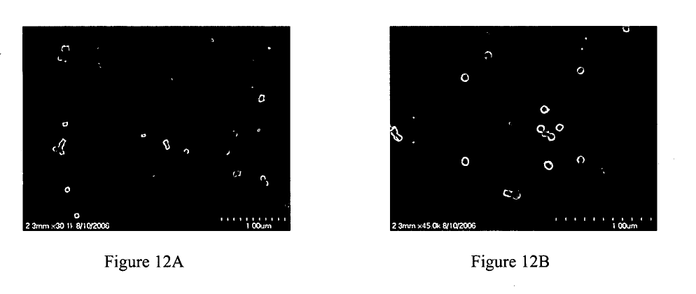

A third particle formulation was also fabricated in the same manner. A degradable monomer, diethyldisulfide methacrylate (84.04%) is blended with (2-acryloxyethyl)trimethyl ammonium chloride (AETMAC) (9.33%). To this monomer blend was added 6.52% by weight 2,2′ diethoxyacetophenone photo-initiator and 0.11 wt % modified antisense oligonucleotide with the sequence CCTCTTACCTCAGTTACAATTTATA (SEQ ID NO: 1) containing a morpholino backbone and a 3′ fluorescein label. The solution was then diluted with 20:1 DMSO:H₂O to enhance miscibility resulting a 73% solids solution. The particles are then fabricated in the same manner as the other degradable particles as described above. FIGS. 12A and 12B show SEM images of the oligo containing particles.

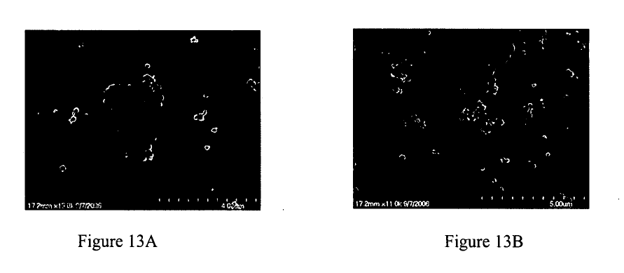

A fourth particle formulation was also fabricated in the same manner. A degradable monomer, diethyldisulfide methacrylate (88.63%) is blended with (2-acryloxyethyl)trimethyl ammonium chloride (AETMAC) (9.53%). To this monomer blend was added 1.51% by weight 2,2′ diethoxyacetophenone photo-initiator and 0.33 wt % modified antisense oligonucleotide with the sequence CCTCTTACCTCAGTTACAATTTATA (SEQ ID NO: 1) containing a morpholino backbone and a 3′ fluorescein label. The solution was then diluted with 3:1 DMF:H₂O to enhance miscibility resulting a 91% solids solution. The particles are then fabricated in the same manner as the other degradable particles as described above. FIGS. 13A and 13B show SEM images of the oligo containing particles.

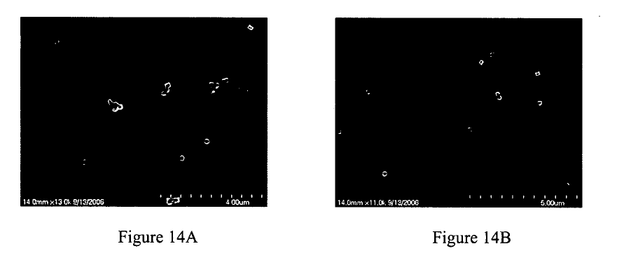

A fifth particle formulation was also fabricated in the same manner. A degradable monomer, diethyldisulfide methacrylate (88.6%) is blended with a tertiary amine monomer to enhance monomer miscibility dimethyl amino ethyl acrylate (10%). To this monomer blend was added 0.5% by weight 4-hydroxyacetophenone photo-initiator and 0.9 wt % modified antisense oligonucleotide with the sequence CTTACCTCAGTTACAATTTATA (SEQ ID NO: 1) containing a morpholino backbone and a 3′ fluorescein label. The solution was then diluted with DMF to enhance miscibility resulting in a 51.4% solids solution. The particles are then fabricated in the same manner as the other degradable particles as described above. FIGS. 14A and 14B show SEM images of the oligo containing particles.

### Example 12

### Encapsulation of Plasmid DNA into Porous Cationic PEG-Diacrylate Particles

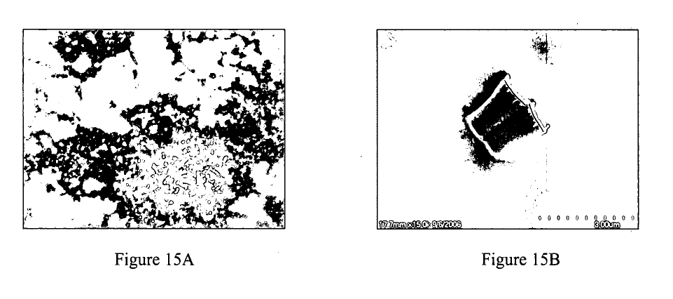

A patterned perfluoropolyether (PFPE) mold was generated by pouring a PFPE-dimethacrylate (PFPE-DMA) containing 1-hydroxycyclohexyl phenyl ketone over a silicon substrate patterned with 1×1×1.8 μm rectangles. The PFPE-DMA covered master was then subjected to UV light (λ=365 nm) for 10 minutes while under a nitrogen purge. The fully cured PFPE-DMA mold was then released from the silicon master. Separately, 10 μg of pCMV Luciferase control plasmid (Elimbiotech) in 12.5 μL of H₂O was added to a mixture of 11.25 mg polyethylene glycol diacrylate (n=9, Polysciences), 1.25 mg of acryloxyethyltrimethylammonium chloride, and 0.25 mg of 4-hydroxyacetophenone. This mixture was spotted directly onto the patterned PFPE-DMA mold and covered with a unpatterned pCTFE film (ACLAR film, 2 mil, Ted Pella, inc). The monomer mixture was pressed between the two polymer sheets, and then the pCTFE was slowly peeled from the patterned PFPE-DMA mold under a saturated H₂O environment to remove any excess monomer solution from the surface of the PFPE-DMA mold. The mold was then subjected to UV light (λ=365 nm) for 2 minutes while maintaining a nitrogen purge that was saturated with H₂O. These particles were harvested by placing a ˜0.4 mL of filtered acetone (0.22 μm PTFE filter) and scrapping the surface of the mold with a glass slide through the drop of acetone. The particle suspension was transferred to a centrifuge tube, the particles were pelleted, the supernatant removed, and the particles were dried under vacuum. The 1×1×1.8 μm particles, as shown in FIGS. 15A and 15B, include 10% cationic, 89.92% PEG-diacrylate (n=9), and 0.08% pCMV Luciferase control vector.

### Example 13

### Encapsulation of Plasmid DNA into Porous PEG-Diacrylate Particles

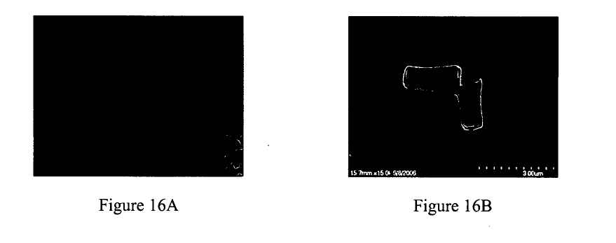

A patterned perfluoropolyether (PFPE) mold was generated by pouring a PFPE-dimethacrylate (PFPE-DMA) containing 1-hydroxycyclohexyl phenyl ketone over a silicon substrate patterned with 1×1×1.8 μm rectangles. The PFPE-DMA covered master was then subjected to UV light (λ=365 nm) for 10 minutes while under a nitrogen purge. The fully cured PFPE-DMA mold was then released from the silicon master. Separately, 10 μg of pCMV Luciferase control plasmid (Elimbiotech) in 12.5 μL of H₂O was added to a mixture of 12.5 mg polyethylene glycol diacrylate (n=9, Polysciences), and 0.25 mg of 4-hydroxyacetophenone. This mixture was spotted directly onto the patterned PFPE-DMA mold and covered with a unpatterned pCTFE film (ACLAR film, 2 mil, Ted Pella, inc). The monomer mixture was pressed between the two polymer sheets, and then the pCTFE was slowly peeled from the patterned PFPE-DMA mold under a saturated H₂O environment to remove any excess monomer solution from the surface of the PFPE-DMA mold. The mold was then subjected to UV light (λ=365 nm) for 2 minutes while maintaining a nitrogen purge that was saturated with H₂O. These particles were harvested by placing a ˜0.4 mL of filtered acetone (0.22 μm PTFE filter) and scrapping the surface of the mold with a glass slide through the drop of acetone. The particle suspension was transferred to a centrifuge tube, the particles were pelleted, the supernatant removed, and the particles were dried under vacuum. The 1×1×1.8 μm particles, as shown in FIGS. 16A and 16B, include 10% cationic, 89.92% PEG-diacrylate (n=9), and 0.08% pCMV Luciferase control vector.

### Example 14

### Encapsulation of Plasmid DNA into Porous Cationic PEG-Diacrylate Particles

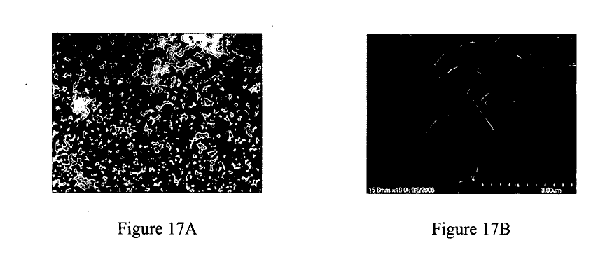

A patterned perfluoropolyether (PFPE) mold was generated by pouring a PFPE-dimethacrylate (PFPE-DMA) containing 1-hydroxycyclohexyl phenyl ketone over a silicon substrate patterned with 1×1×1.8 μm rectangles. The PFPE-DMA covered master was then subjected to UV light (λ=365 nm) for 10 minutes while under a nitrogen purge. The fully cured PFPE-DMA mold was then released from the silicon master. Separately, a mixture of 11.25 mg polyethylene glycol diacrylate (n=9, Polysciences), 1.25 mg of acryloxyethyltrimethylammonium chloride, and 0.25 mg of 4-hydroxyacetophenone was prepared. This mixture was spotted directly onto the patterned PFPE-DMA mold and covered with a unpatterned pCTFE film (ACLAR film, 2 mil, Ted Pella, inc). The monomer mixture was pressed between the two polymer sheets, and then the pCTFE was slowly peeled from the patterned PFPE-DMA mold under a saturated H₂O environment to remove any excess monomer solution from the surface of the PFPE-DMA mold. The mold was then subjected to UV light (λ=365 nm) for 2 minutes while maintaining a nitrogen purge that was saturated with H₂O. These particles were harvested by placing a ˜0.4 mL of filtered acetone (0.22 μm PTFE filter) and scrapping the surface of the mold with a glass slide through the drop of acetone. The particle suspension was transferred to a centrifuge tube, the particles were pelleted, the supernatant removed, and the particles were dried under vacuum. The 1×1×1.8 μm particles, as shown in FIGS. 17A and 17B, include 10% cationic, 89.92% PEG-diacrylate (n=9), and 0.08% pCMV Luciferase control vector.

### Example 15

### Transfection of HeLa Cells with 1 μm×1 μm×1.8 μm pCMV Luciferase Porouse PEG PRINT Particles

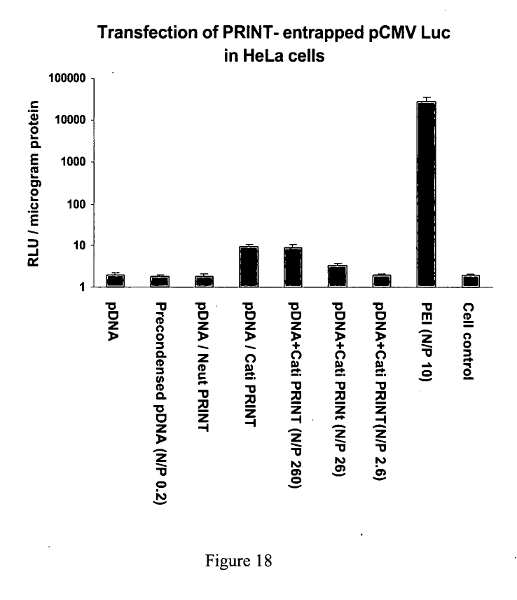

The porouse pDNA containing PEG particles fabricated using PRINT were dispersed such that the pDNA concentration was 1 μg/200 μL of water (1.25 mg particles/200 L H₂O) to be used in cellular uptake experiments. A comparable amount of cationic particles without plasmids were used as a control and as condensation agents for the delivery of free plasmid. These particles were exposed to 10⁵ HeLa cells at a final concentration of particles of 310 μg/mL in serum-free OptiMEM media. The particles and cells were incubated for 4 hrs at 5% CO₂ at 37° C. The particles were washed from the cells using OptiMEM w/10% FBS, and the 800 μL of fresh media was placed on the cells. The cells were then incubated for an additional 44 hours, lysed, and treated with Luciferin. The bioluminescence from the produced Luciferase was measured using a luminescence plate reader, as shown in FIG. 18. The total luminescence was normalized relative to the total protein concentrations per well using the BSA protein assay. Cationic PRINT particles containing pCMV luciferase show ˜5-fold increase of luminescence relative to background, indicating successful delivery of the pDNA to the cell.

### Example 16

### Fabrication of ssDNA Containing Cationic Disulfide PRINT Particles

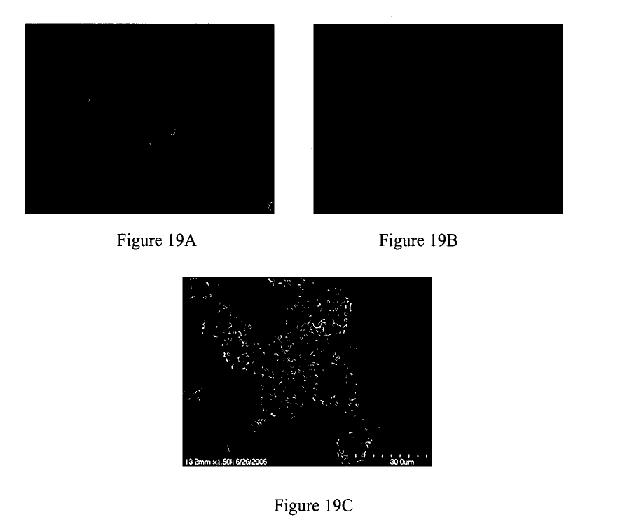

A patterned perfluoropolyether (PFPE) mold was generated by pouring a PFPE-dimethacrylate (PFPE-DMA) containing 1-hydroxycyclohexyl phenyl ketone over a silicon substrate patterned with 2×2×1 μm rectangles. The PFPE-DMA covered master was then subjected to UV light (λ=365 nm) for 10 minutes while under a nitrogen purge. The fully cured PFPE-DMA mold was then released from the silicon master. Separately, 2 μg of ssDNA (18mer, sequence GCT ATT ACC TTA ACC CAG (SEQ ID NO: 2) containing a 3′ fluorescein label) in 2 μL of H₂O was added to a mixture of 13.65 mg of bis(ethyl methacrylate)disulfide, 1.53 mg of acryloxyethyltrimethylammonium chloride, 0.075 mg of Poly 0.15 mg 2,2′-diethoxyacetophenone, 2.34 mg of acetonitrile, 2.34 mg of methanol, 9.5 mg of N,N-dimethylformamide, and 0.4 mg of H₂O. This mixture was spotted directly onto the patterned PFPE-DMA mold and covered with a unpatterned pCTFE film (ACLAR film, 2 mil, Ted Pella, inc). The monomer mixture was pressed between the two polymer sheets, and then the pCTFE was slowly peeled from the patterned PFPE-DMA mold under a saturated H₂O environment to remove any excess monomer solution from the surface of the PFPE-DMA mold. The mold was then subjected to UV light (λ=365 nm) for 2 minutes while maintaining a nitrogen purge that was saturated with N,N-dimethylformamide. These particles were harvested by placing a ˜0.4 mL of filtered acetone (0.22 μm PTFE filter) and scrapping the surface of the mold with a glass slide through the drop of acetone. The particle suspension was transferred to a centrifuge tube, the particles were pelleted, the supernatant removed, and the particles were dried under vacuum, as shown in FIGS. 19A-19C. The particles (1.44 mg) were then suspended in 1 mL of H₂O, vortex rigorously, and pellet out to remove any loosely associated ssDNA. The supernatant was removed for analysis of ssDNA in solution by total fluorescence of the FITC label (excitation 488 nm, emission 515 nm) relative to a calibration curve of the fluorescence of a known concentration of identical 3′-label fluorescein ssDNA. The remaining pellet was dried under vacuum. The final weight percent of ssDNA in particles was 0.008%.

### Example 17

### Release of ssDNA from Disulfide PRINT Particles Under Reductive Conditions

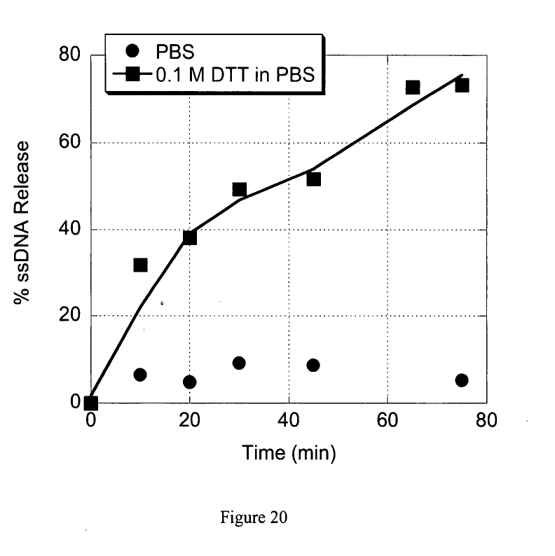

1.5 mg of 2×2×1 μm particles composed of 0.008% ssDNA (18mer, GCT ATT ACC TTA ACC CAG (SEQ ID NO: 2) containing a 3′ fluorescein label), 89% bis(ethyl methacrylate)disulfide, 9.9% of acryloxyethyltrimethylammonium chloride, 1% 2,2′-diethoxyacetophenone, and 0.05% Polyfluor 570 were suspended in PBS (pH 7.4) at a concentration of 0.3 mg/mL. The suspension was divided into two tubes fitted with magnetic stirbars (2.5 mL each). To one tube 38.5 mg of dithiothreitol (0.1 M) was added, and both tubes were stirred in the dark. Samples were withdrawn from each tube at given times and filtered through 0.2 μm filters. The concentration of release oligo in the filtrate was determined by the total fluorescence of each sample (excitation 488 nm, emission 515 nm) relative to a calibration curve of the fluorescence of a known concentration of identical 3′-label fluorescein ssDNA. The relative amount of ssDNA release was calculated from the observed concentration divided by the total concentration of ssDNA in the reaction prior to filtration (release ssDNA plus unreleased ssDNA). Oligonucleotide is release rapidly in the presence of dithiothreitol within 80 minutes, but little release is observed in the absence of reductant, as shown in FIG. 20.

### Example 18

### Proposed Synthesis of Hydrophilic Disulfide Crosslinkers

The bis(ethylene methacrylate) disulfide is an effect crosslinker reduction activated release of biological cargos, however, its inherent hydrophobicity prevents the simple incorporation of high levels of biomolecules. The following disulfide crosslinkers should provide the same reactivity, but should be freely soluble in H₂O solutions with biomolecular cargos.

Ostensibly, the reaction of the N-termini of oxidized glutathione with 2-isocyanatoethylmethacrylate should yield a bis-methacrylate crosslinker that contains a disulfide unit between the two tripeptide chains. The ionic nature of hexapeptide crosslinker should provide a greater degree of hydrophillicity for incorporation of hydrophilic cargos. Alternatively, glycidyl methacrylate and cystamine are reacted to form the epoxide opened product, which is then protonated to form the hydrochloride salt. This crosslinking agent should be reduction activated and freely soluble in H₂O (Scheme 3).

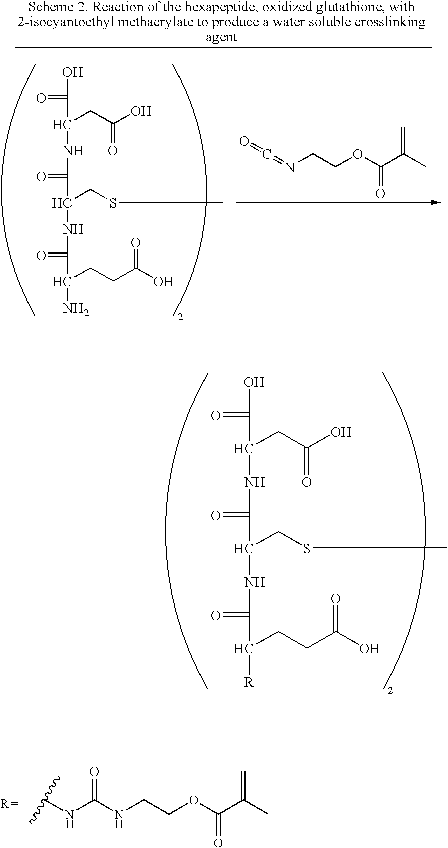

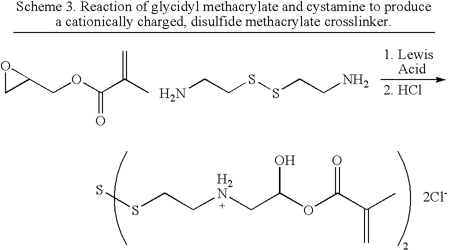

### Example 19

### Synthesis of Degradable Crosslinkers for Hydrolysable Print Particles

Bis(ethylene methacrylate)disulfide (DEDSMA) was synthesized using methods described in Li et al. Macromolecules 2005, 38, 8155-8162 from 2-hyrdoxyethane disulfide and methacroyl chloride (Scheme 1). Analogously, bis(8-hydroxy-3,6-dioxaoctyl methacrylate) disulfide (TEDSMA) was synthesized from bis(8-hydroxy-3,6-dioxaoctyl)disulfide (Lang et al. Langmuir 1994, 10, 197-210). Methacroyl chloride (0.834 g, 8 mmole) was slowly added to a stirred solution of bis(8-hydroxy-3,6-dioxaoctyl)disulfide (0.662 g, 2 mmole) and triethylamine (2 mL) in acetonitrile (30 mL) chilled in an ice bath. The reaction was allowed to warm to room temperature and stirred for 16 hours. The mixture was diluted with 5% NaOH solution (50 mL) and stirred for an additional hour. The mixture was extracted with 2×60 mL of methylene chloride, the organic layer was washed 3×100 mL of 1 M NaOH, dried with anhydrous K₂CO₂, and filtered. Removal of the solvent yielded 0.860 g of the TEDSMA as a pale yellow oil. ¹H NMR (CDCl₃) δ=6.11 (2H, s), 5.55 (2H, s), 4.29 (4H, t), 3.51-3.8 (16H, m), 2.85 (4H, t), 1.93 (6H, s).

### Targeting PRINT Particles Through Surface Functionalization

### Example 20

### Preparation of Polyethylene Glycol Carbonylimidizole Monomethacrylate

Polyethylene glycol monomethacrylate (5.0 g, n=400, Polysciences) was dissolved in 100 mL CHCl₃ followed by the addition of 5.0 g carbonyldiimidazole and the reaction was stirred for 16 h. The organic phase was washed repeatedly with chilled water until the pH of the aqueous rinse became neutral. The organic phase was then dried over MgSO₄, filtered, and the solvent removed by rotovap leaving 5.7 g of a colorless oil. ¹H NMR (CDCl₃) δ=8.15 (1H, d), 7.45 (1H, d), 7.05 (1H, s), 6.11 (1H, s), 5.57 (1H, s), 4.60-4.30 (4H, m), 3.85-3.60 (30H, m), 1.95 (3H, s).

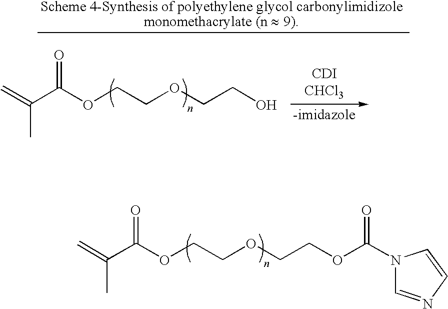

### Example 21

### Preparation of Cylindrical Particles Having Streptavidin (Alexa Fluor 488) Conjugated to Their Surfaces

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned PE film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial.

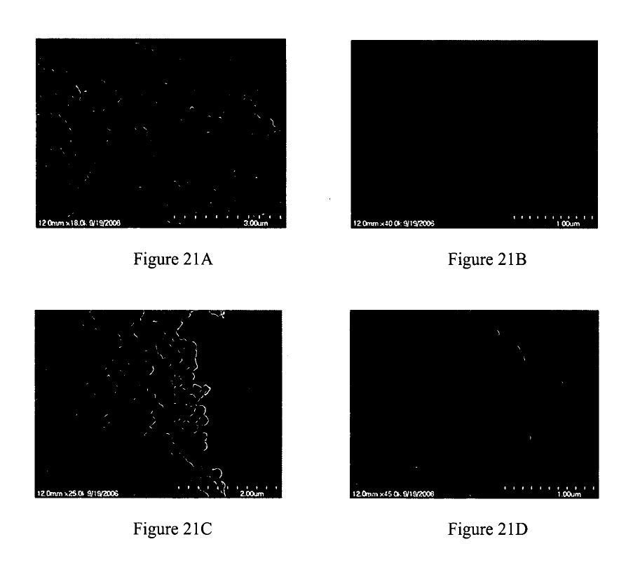

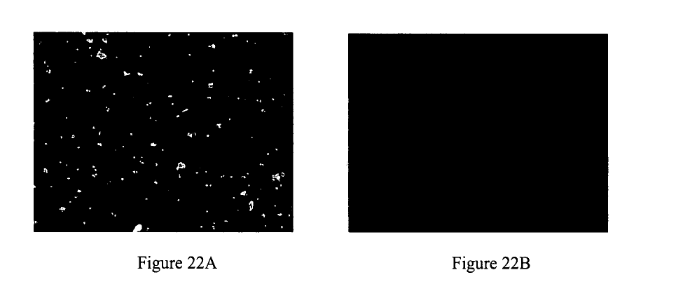

300 μL of a 2 mg/mL streptavidin solution in PBS (Alexa Fluor 488, Invitrogen) was diluted to 600 μL with PBS. This solution was added directly to a suspension of 200 nm tall×200 nm diameter cylindrical particles with carbonylimidazole on the surface in 12 mL of DMSO. The solution was stirred for 14 h at rt and then diluted to 30 mL with deionized water. The particles were collected on a cetrifugal filter membrane (Millipore, PVDF membrane, 100 nm pore size). They were resuspended in 2 mL of water and filtered (Fisher brand P8, 20-25 μm pore size). The particles were again collected on a cetrifugal filter membrane (Millipore, PVDF membrane, 100 nm pore size) and then resuspended in 3.0 mL of water. 10 μL of the solution was spotted onto a glass slide and the dried under vacuum, as shown in the SEM images of FIGS. 21A-21D and the DIC and fluorescence images of FIGS. 22A and 22B.

### Example 22

### Preparation of Particles Having Biotin Anti-Mouse CD11b Conjugated to Their Surfaces

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned PE film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial.

300 μL of a 2 mg/mL streptavidin solution in PBS (Alexa Fluor 488, Invitrogen) was diluted to 600 μL with PBS. This solution was added directly to a suspension of 200 nm tall×200 nm diameter cylindrical particles with carbonylimidazole on the surface in 12 mL of DMSO. The solution was stirred for 14 h at rt and then diluted to 30 mL with deionized water. The particles were collected on a cetrifugal filter membrane (Millipore, PVDF membrane, 100 nm pore size). They were resuspended in 2 mL of water and filtered (Fisher brand P8, 20-25 μm pore size). The particles were again collected on a cetrifugal filter membrane (Millipore, PVDF membrane, 100 nm pore size) and then resuspended in 3.0 mL of water. 500 μL of the particle solution was removed and placed in an effendorf tube. 100 μg of biotinylated anti-mouse CD11b (in 100 μL PBS, eBiosciences) was added to the 200 nm particle solution. The solution was stored at 4° C.

### Example 23

### Preparation of Particles Having Biotin Anti-Mouse CD11c Conjugated to Their Surfaces

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned PE film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial.

300 μLof a 2 mg/mL streptavidin solution in PBS (Alexa Fluor 488, Invitrogen) was diluted to 600 μL with PBS. This solution was added directly to a suspension of 200 nm tall×200 nm diameter cylindrical particles with carbonylimidazole on the surface in 12 mL of DMSO. The solution was stirred for 14 h at rt and then diluted to 30 mL with deionized water. The particles were collected on a cetrifugal filter membrane (Millipore, PVDF membrane, 100 nm pore size). They were resuspended in 2 mL of water and filtered (Fisher brand P8, 20-25 μm pore size). The particles were again collected on a cetrifugal filter membrane (Millipore, PVDF membrane, 100 nm pore size) and then resuspended in 3.0 mL of water. 500 μL of the particle solution was removed and placed in an effendorf tube. 100 μg of biotinylated anti-mouse CD11c (in 100 μL PBS, eBiosciences) was added to the 1.2 mg/mL solution of 200 nm tall×200 nm diameter cylindrical particles coated with streptavidin (Alexa Fluor 488). The solution was stored at 4° C.

### Example 24

### Preparation of Particles Having Biotin Anti-Mouse CD80 Conjugated to Their Surfaces

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned PE film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial. 300 μL of a 2 mg/mL streptavidin solution in PBS (Alexa Fluor 488, Invitrogen) was diluted to 600 μL with PBS. This solution was added directly to a suspension of 200 nm tall×200 nm diameter cylindrical particles with carbonylimidazole on the surface in 12 mL of DMSO. The solution was stirred for 14 h at rt and then diluted to 30 mL with deionized water. The particles were collected on a cetrifugal filter membrane (Millipore, PVDF membrane, 100 nm pore size). They were resuspended in 2 mL of water and filtered (Fisher brand P8, 20-25 μm pore size). The particles were again collected on a cetrifugal filter membrane (Millipore, PVDF membrane, 100 nm pore size) and then resuspended in 3.0 mL of water. 500 μL of the particle solution was removed and placed in an effendorf tube, and 100 μg of biotinylated anti-mouse CD80 (in 100 μL PBS, eBiosciences) was added. The solution was stored at 4° C.

### Example 25

### Preparation of Particles Having Biotin Anti-Human CD11b Conjugated to Their Surfaces

A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned PE film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial.

300 μL of a 2 mg/mL streptavidin solution in PBS (Alexa Fluor 488, Invitrogen) was diluted to 600 μL with PBS. This solution was added directly to a suspension of 200 nm tall×200 nm diameter cylindrical particles with carbonylimidazole on the surface in 12 mL of DMSO. The solution was stirred for 14 h at rt and then diluted to 30 mL with deionized water. The particles were collected on a cetrifugal filter membrane (Millipore, PVDF membrane, 100 nm pore size). They were resuspended in 2 mL of water and filtered (Fisher brand P8, 20-25 μm pore size). The particles were collected on a cetrifugal filter membrane (Millipore, PVDF membrane, 100 nm pore size) and then resuspended in 3.0 mL of water. 100 μg of biotinylated anti-human CD11b (in 100 μL PBS, eBiosciences) was added to 500 μL of a 1.2 mg/mL solution of 200 nm diameter cylindrical particles coated with streptavidin (Alexa Fluor 488). The solution was stored at 4° C.

### Encapsulation of Anti-Luc siRNA in PRINT Particles

### Example 26

### Fabrication of PEG Based 1 μm×1 μm×0.6 μm Particles with Fluorescently Tagged Anti-Luc siRNA

A 6 inch silicon substrate patterned with 1 μm×1 μm×0.6 μm rounded edge box shapes is placed under a UV source. A patterned perfluoropolyether (PFPE) mold is generated by pouring 11 mL of PFPE-dimethacrylate (PFPE-DMA) containing 0.1 wt % 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

Separately, a poly(ethylene glycol) (PEG) diacrylate (n=9) (87.9%) is blended with (2-acryloxyethyl)trimethyl ammonium chloride (AETMAC) (9.7%). To this monomer blend was added 1.0% by weight 2,2diethoxyacetophenone photo-initiator and 1.4 wt % Anti-Luc siRNA labeled with NuLight DY-547 Phosphoramidite (siRNA purchased from Dharmacon). The solution was then diluted with water to enhance miscibility resulting in a 54% solids solution. Following this, 8 μL of the above monomer blend is evenly spotted onto the PFPE-DMA mold. A chlorotrifluoroethanol (CTFE) film measuring 8.5×11 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber was purged with Nitrogen blowing through a gas scrubber containing water for 10 minutes and then exposed to UV light (λ=365 nm) at 2-4 mW/cm² for 10 minutes.

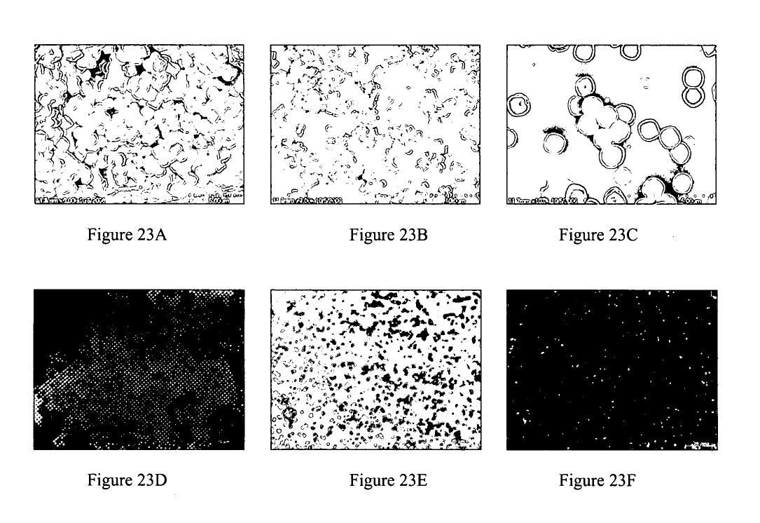

The cured mold is then removed from the UV chamber. 300 μL of acetone that has been filtered through a PTFE membrane (0.22 μm) is placed atop the mold. A glass slide is used to scrape the solvent across the mold and release the particles, turning the acetone cloudy and colored. The scraping of the mold continues for several passes until no more particles are released. The particles are then imaged by optical, fluorescent microscopy and SEM, as shown in FIGS. 23A-23F.

### Encapsulation of 6-ROX 2′O-MOE Antisense Oligonucleotide in Print Particles

### Example 27

### Fabrication of PEG Based 1 μm×1 μm×0.6 μm Round Boxes with Fluorescently Tagged Anti-Luc siRNA

A 6 inch silicon substrate patterned with 2 μm×2 μm×1 μm box shapes is placed under a UV source. A patterned perfluoropolyether (PFPE) mold is generated by pouring 11 mL of PFPE-dimethacrylate (PFPE-DMA) containing 0.1 wt % 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

Separately, a poly(ethylene glycol) (PEG) diacrylate (n=9) (98.985%) is blended with 1.0% by weight 2,2′-diethoxyacetophenone photo-initiator and 0.015 wt % 6 ROX-2′O-MOE (red fluorophore). 6 μL of the above monomer blend is evenly spotted onto the PFPE-DMA mold. A polyethylene film measuring 8×10 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber was purged with Nitrogen for 2 minutes and then exposed to UV light (λ=365 nm) at 20 mW/cm² for 3 minutes.

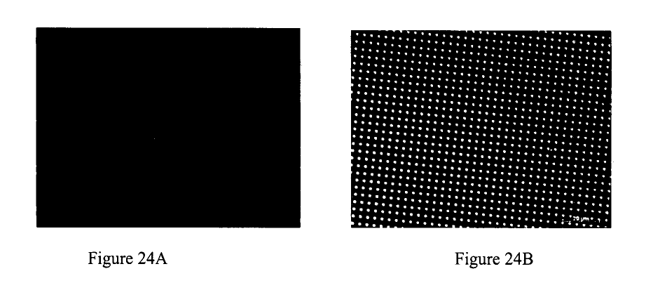

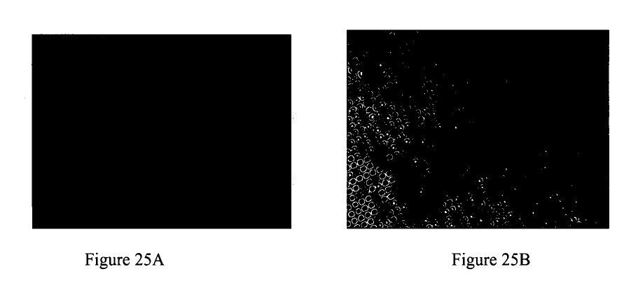

The cured mold is then removed from the UV chamber. The particles still in the mold are imaged by fluorescent and optical microscopy, as shown in FIGS. 24A and 24B respectively. A portion of the mold was harvested with cyanoacrylate and imaged by fluorescent and optical microscopy, as shown in FIGS. 25A and 25B respectively.

### Example 28

### Synthesis of Anisamide-Based Targeting Ligand

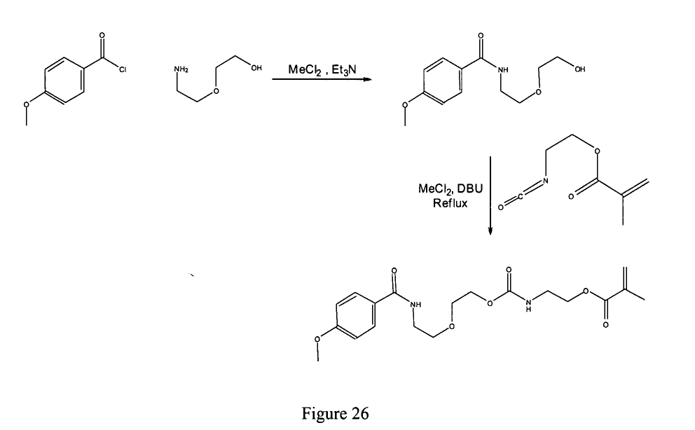

The targeting of sigma-receptor bearing cells has been accomplished using anisamide containing targeting ligands in liposomes (Huang et. Al, 2004). A similar target ligand can be synthesized for use in PRINT-based nanoparticles, as shown in FIG. 26. A solution of p-anisoyl chloride (1.0 g, 0.00586 mole) in 10 mL of methylene chloride was slowly dripped in to a solution of 2-(2-aminoethoxy)ethanol (0.6155 g, 0.00586 mole) and triethylamine (2 mL) in 10 mL of methylene chloride cooled in an ice bath. The reaction was allowed to warm to room temperature and stirred for 3 hours. The reaction was diluted with 20 mL of methylene chloride and then washed with 40 mL of 1 M NaOH three times. The organic layer was dried with potassium carbonate, filtered, and the solvent was removed to yield a light yellow oil (0.8 g, 60%). The oil was then used directly without further purification.

The above oil (0.8 g, 0.00334 mole) was dissolved in 20 mL of methylene chloride in a round bottom flask. To this solution 2-isocyanatoethyl methacrylate (0.570 g, 0.0037 mole) and 1,8-Diazabicyclo[5.4.0]undec-7-ene (0.05 g, 0.33 mmole), the flask fitted with a condensor, and heated to reflux for 3 hours. The solution was cooled to room temperature and then washed with 20 mL of 0.5 M HCl two times and then with 20 mL of 1 M NaOH two times. The organic layer was dried with potassium carbonated, filtered, and the solvent removed under vacuum, yielding a light yellow oil (1.13 g, 88%). ¹H NMR (CDCl₃) δ=7.76 (d, 2H), 6.91 (d, 2H), 6.56 (s, 1H), 6.08 (s, 1H), 5.56 (s, 1H), 5.05 (s, 1H), 4.21 (m, 4H), 3.82 (s, 3H), 3.64 (m, 6H), 3.42 (m, 2H), 1.91 (s, 3H).

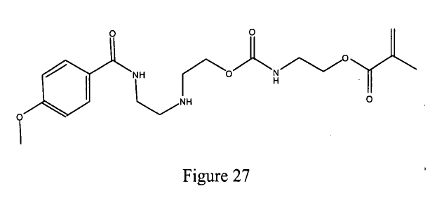

Alternatively, 2-(2-aminoethylamino)ethanol can be used instead of 2-(2-aminoethoxy)ethanol in the initial step of the synthesis to produce the target ligand, as shown in FIG. 27, which may have preferable targeting properties towards sigma receptor mediated endocytosis.

### Example 29

### Fabrication of Degradable 200 nm Diameter Cylinders (200 nm Height) with Proton Sponge Monomer and Fluorescently Tagged Anti-Luciferase siRNA Cargo

An 8 inch silicon substrate patterned with 200 nm tall×200 nm diameter cylindrical shapes is placed under a UV source. A patterned perfluoropolyether (PFPE) mold is generated by pouring 20 mL of PFPE-dimethacrylate (PFPE-DMA) containing 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

Separately a solution was prepared containing 50 ug of anti-luciferase siRNA in water (Dy-547 labeled on the 5′ end of the sense strand) and AETMAC giving an N/P ratio of 5. To this mixture is added by weight a degradable monomer diethyldisulfide methacrylate, 2,2-diethoxyacetophenone photo initiator, and N-morpholinoethyl acrylate. The final solution contained 0.5% anti-luciferase siRNA, 1.5% AETMAC, 76% diethyldisulfide methacrylate, 2% 2,2-diethoxyacetophenone photo initiator, and 20% 2-N-morpholinoethyl acrylate. This solution was then diluted with DMSO to enhance miscibility resulting in a 40% solids solution. Following this, 15 μL of the above monomer blend is evenly spotted onto the PFPE-DMA mold. A chlorotrifluoroethylene film measuring 8.5×11 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber is purged with Nitrogen for 2 minutes and then exposed to UV light at greater than 20 mW/cm² for 3 minutes.

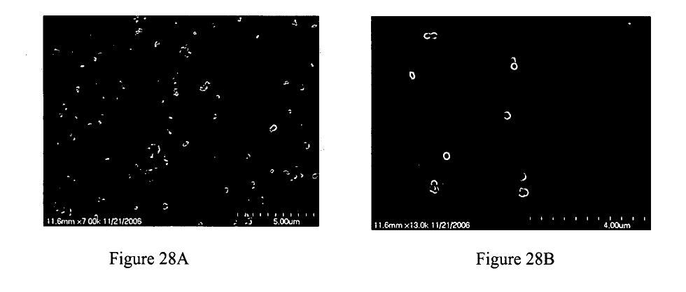

The cured mold is then removed from the UV chamber. 300 μL of doubly distilled filtered (0.22 μm) water is placed atop the mold. A glass slide is used to scrape the water across the mold and release the particles into the water, turning the water cloudy. The scraping of the mold continues for several passes until no more particles are released. The cloudy water suspension is then collected and filtered thru a 20 μm filter. The filtrate is then collected and concentrated using a 0.1 μm centricon tube. Finally, the particles are resuspended from the filter into pure water. SEM images of the particles are shown in FIGS. 28A and 28B:

### Example 30

### Fabrication of PEG Based 200 nm Diameter Cylinders (200 nm Height) with Proton Sponge Monomer and Fluorescently Tagged Anti-Luciferase siRNA Cargo

An 8 inch silicon substrate patterned with 200 nm tall×200 nm diameter cylindrical shapes is placed under a UV source. A patterned perfluoropolyether (PFPE) mold is generated by pouring 20 mL of PFPE-dimethacrylate (PFPE-DMA) containing 2,2-diethoxyacetophenone onto the patterned silicon substrate. The UV source containing the silicon substrate covered with PFPE-DMA is flushed with nitrogen for 2 minutes to remove oxygen. The apparatus is then subjected to UV light (λ=365 nm) for 3 minutes. The fully cured PFPE-DMA mold is then released from the silicon master in the mold maker.

Separately a solution was prepared containing 0.5 wt % anti-luciferase siRNA in water (Dy-547 labeled on the 5′ end of the sense strand), 88.9 wt % PEG diAcrylate, 0.2 wt % 2,2-diethoxyacetophenone photo initiator, and 10.1% 2-N-morpholinoethyl acrylate. This solution was then diluted with H₂O to enhance miscibility resulting in a 50% solids solution. Following this, 15 μL of the above monomer blend is evenly spotted onto the PFPE-DMA mold. A chlorotrifluoroethylene film measuring 8.5×11 inches is placed atop the mold. Pressure is applied with a roller for a few strokes to help spread the monomer solution. The film is removed, turned slightly and placed again atop the mold. Again, pressure is applied with a roller to help spread the monomer. This turning of the film and spreading of the monomer solution is repeated until the entire mold is covered with the solution. After spreading, the film remains atop the mold. Then, the film is slowly peeled back from the surface of the mold resulting in filling of only the depressions in the mold. The filled mold is then placed in a UV chamber. The chamber is purged with Nitrogen for 2 minutes and then exposed to UV light at greater than 20 mW/cm² for 3 minutes.

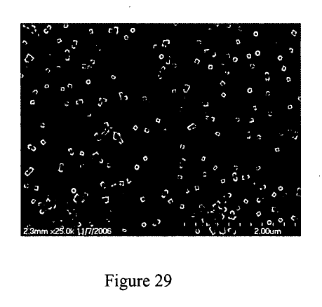

The cured mold is then removed from the UV chamber. 300 uL of doubly distilled filtered (0.22 um) water is placed atop the mold. A glass slide is used to scrape the water across the mold and release the particles into the water, turning the water cloudy. The scraping of the mold continues for several passes until no more particles are released. The cloudy water suspension is then collected and filtered thru a 20 um filter. The filtrate is then collected and concentrated using a 0.1 um centricon tube. Finally, the particles are resuspended from the filter into pure water. An SEM image of the particles is shown in FIG. 29.

### Example 31

### Preparation of Disulfide-Based Cylindrical PRINT™ Particles with Avidin-Functionalized Surfaces Containing Fluorescein-o-Acrylate

A nano-cavity mold was generated by pouring FLUOROCUR™ material (Liquidia Technologies, Inc., North Carolina) over a silicon substrate patterned with 200 nm tall×200 nm diameter cylinders. The FLUOROCUR material covered master was then subjected to UV light (λ=365 nm) for 3 minutes while under a nitrogen purge. The fully cured FLUOROCUR based mold was then released from the silicon master. Separately, a mixture of 68 mg trimethylolpropane ethoxylate triacrylate (˜900 MW), 20 mg cystaminebisacrylamide, 10 mg 2-amino-ethyl-methacrylate, 1 mg fluorescein-o-acrylate, 1 mg 1-hydroxycyclohexylphenyl ketone was prepared in 50 μL DMSO. This mixture was spotted directly onto the patterned Fluorocur based mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 2 mL of chloroform on the mold and scraping the surface with a glass slide. The particle suspension was transferred to a scintillation vial.

Five molds of particles with the composition listed above were harvested mechanically into a total of 17 mL of chloroform. NHS-PEO₁₂-Biotin (250 μL, 42 mg/mL in DMSO) was added and the mixture was stirred for 3.5 h. Acetic anhydride (50 μL) was added and the mixture was stirred for 0.5 h. Particles were purified by vacuum filtration (P8, Fisherbrand) and collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water.

UltraAvidin (2 mL, 2.5 mg/mL in water) was added to the particle solution from above. After stirring for 18 h, particles were collected onto a centrifugal filter membrane (0.1 μm pore size, PVDF membrane, Millipore) and then washed with 15 mL of water to remove any unbound avidin. The particles were resuspended in 1.2 mL of water. The solution was analyzed by TGA, DLS, zeta potential, and SEM. The remaining particle solution was concentrated to 0.5 mL of a 2.8 mg/mL solution by centrifugation (12,000 rpm, 2 min). The particles were spun down into a pellet and the desired amount of supernatant removed.

Titration of available biotin binding sites: Available biotin binding sites were quantified by fluorescence spectroscopy. UltraAvidinated PRINT particles (50 μL, 1.4 mg/mL) were added to a solution of biotin-4-fluorescein (90 nM in water) and the mixture was stirred for 10 min. Particles were removed from solution by filtration (0.1 μm pore size, PVDF membrane, Millipore) and the concentration of biotin-4-fluorescein remaining in solution was then determined. The decrease in concentration of biotin-4-fluorescein can be attributed to binding to UltraAvidinated PRINT particles (and thus removal from solution by filtration). Results translated to ˜5500 binding sites/particle based on an average particle weight of 6.91×10⁻¹⁵. The number of copies of UltraAvidin/particle was in the range 1,833-5,500 depending on the number of biotin binding sites occupied during attachment to the particle. The low value assumes 3 binding sites were occupied by attachment to the particle whereas the high number assumes only one site was used.

### Example 32

### Preparation of Disulfide-Based Cylindrical PRINT Particles with Avidin-Functionalized Surfaces Containing Dexamethasone

A nano-cavity mold was generated by pouring FLUOROCUR™ material over a silicon substrate patterned with 200 nm tall×200 nm diameter cylinders. The Fluorocur material covered master was then subjected to UV light (λ=365 nm) for 3 minutes while under a nitrogen purge. The fully cured Fluorocur based mold was then released from the silicon master. Separately, a mixture of 67 mg trimethylolpropane ethoxylate triacrylate (˜900 MW), 20 mg cystaminebisacrylamide, 10 mg 2-amino-ethyl-methacrylate, 2 mg dexamethasone, 1 mg 1-hydroxycyclohexylphenyl ketone was prepared in 50 μL DMSO. This mixture was spotted directly onto the patterned mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 2 mL of chloroform on the mold and scraping the surface with a glass slide. The particle suspension was transferred to a scintillation vial.

Five molds of particles with the composition listed above were harvested into 16 mL of chloroform. NHS-PEO₁₂-Biotin (250 μL, 50 mg/mL in DMSO) was added and the mixture was stirred for 20 h. Acetic anhydride (200 μL) was added and the mixture was stirred for 1 h. Particles were collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water. DMSO (2 mL) was then added.

UltraAvidin (1 mL, 2.5 mg/mL in water) was added to the particle solution from above. After stirring for 18 h, particles were diluted with 10 mL of water, purified by vacuum filtration (P8, Fisherbrand), and pelletized from the filtrate using centrifugation (8500 rpm, 50 mL falcon tube). The supernatant was removed and the particles were re-suspended in 30 mL of water. The particles were again pelletized and the supernatant removed leaving approx. 5 mL of water. Particles were collected from the remaining solution onto a centrifugal filter membrane (0.1 μm pore size, PVDF membrane, Millipore) and then washed with 15 mL of water to remove any unbound avidin. The particles were re-suspended in 1.2 mL of water. The solution was analyzed by TGA, DLS, zeta potential, and SEM. The remaining particle solution was concentrated to 0.5 mL of a 2.3 mg/mL solution by centrifugation (10,000 rpm, 2 min). The particles were spun down into a pellet and the desired amount of supernatant removed.

### Example 33

### Preparation of Disulfide-Based Square PRINT Particles with Avidin-Functionalized Surfaces Containing Doxorubicin for Release Studies

A patterned micro-cavity mold was generated by pouring FLUOROCUR™ material over a silicon substrate patterned with 2×2×2 μm squares. The FLUOROCUR material covered master was then subjected to UV light (λ=365 nm) for 3 minutes while under a nitrogen purge. The fully cured Fluorocur based mold was then released from the silicon master. Separately, a mixture of 57 mg trimethylolpropane ethoxylate triacrylate (˜900 MW), 30 mg cystaminebisacrylamide, 10 mg 2-amino-ethyl-methacrylate, 2 mg doxorubicin, 1 mg 1-hydroxycyclohexylphenyl ketone was prepared in 50 μL DMSO. This mixture was spotted directly onto the patterned Fluorocur based mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 2 mL of chloroform on the mold and scraping the surface with a glass slide. The particle suspension was transferred to a scintillation vial.

NHS-PEO₁₂-Biotin (250 μL, 42 mg/mL in DMSO) was added and the mixture was stirred for 24 h. Acetic anhydride (50 μL) was added and the mixture was stirred for 1 h. Particles were purified by vacuum filtration (P8, Fisherbrand) and collected from solution onto a centrifugal filter membrane (0.1 μm pore size, PVDF membrane, Millipore). Excess NHS-PEO₁₂-Biotin and acetic anhydride were removed by thorough washing with chloroform (10 mL). The particles were re-suspended in 5 mL ultra pure water. UltraAvidin (2 mL, 2.5 mg/mL in water) was added to the particle solution from above. After stirring for 24 h, particles were collected onto a centrifugal filter membrane (0.1 μm pore size, PVDF membrane, Millipore) and then washed with 15 mL of water to remove any unbound avidin. The particles were re-suspended in 10 mL PBS.

The 10 mL particle solution from above was divided into two aliquots. Dithiothreitol (DTT, 550 μL of a 1 M solution in water) was added to one aliquot and both samples were stirred and monitored by flow cytometry over 48 h. Release of the fluorophore (doxorubicin) was clearly dependent on the presence of a reducing agent, as shown in FIG. 30.

### Example 34

### Preparation of Disulfide-Based Square PRINT Particles with Amine-Functionalized Surfaces Containing Doxorubicin for Cell Viability Studies

A patterned micro-cavity mold was generated by pouring FLUOROCUR™ material over a silicon substrate patterned with 2×2×2 μm squares. The FLUOROCUR material covered master was then subjected to UV light (λ=365 nm) for 3 minutes while under a nitrogen purge. The fully cured Fluorocur based mold was then released from the silicon master. Separately, a mixture of 57 mg trimethylolpropane ethoxylate triacrylate (˜900 MW), 30 mg cystaminebisacrylamide, 10 mg 2-amino-ethyl-methacrylate, 2 mg doxorubicin, 1 mg 1-hydroxycyclohexylphenyl ketone was prepared in 50 μL DMSO. This mixture was spotted directly onto the patterned FLUOROCUR based mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 2 mL of chloroform on the mold and scraping the surface with a glass slide. The particles were collected from solution onto a centrifugal filter membrane (0.65 μm pore size, PVDF membrane, Millipore), weighed, and re-suspended in water. Particles were dosed on HeLa cells (50,000 per well/96 well plate) and incubated for 72 h. Cell viability was then assayed using CyQuant dye. Doxorubicin release in vitro is given as a function of cell viability in FIG. 31.

### Example 35

### Preparation of Disulfide-Based Square PRINT Particles with Amine-Functionalized Surfaces Containing Rhodamine-B

A patterned FLUOROCUR™ based mold was generated by pouring FLUOROCUR material over a silicon substrate patterned with 3×3×3 μm squares. The FLUOROCUR material covered master was then subjected to UV light (λ=365 nm) for 3 minutes while under a nitrogen purge. The fully cured Fluorocur based mold was then released from the silicon master. Separately, a mixture of 67 mg trimethylolpropane ethoxylate triacrylate (˜900 MW), 20 mg cystaminebisacrylamide, 10 mg 2-amino-ethyl-methacrylate, 2 mg rhodamine-B, 1 mg 1-hydroxycyclohexylphenyl ketone was prepared in 50 μL DMSO. This mixture was spotted directly onto the patterned Fluorocur based mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 2 mL of chloroform on the mold and scraping the surface with a glass slide. The particle suspension was transferred to a scintillation vial.

### Example 36

### Targeting T Cells Using Disulfide-Based Cylindrical Particles with MHC-Antigen/Avidin-Functionalized Surfaces Containing Fluorescein-o-Acrylate

Avidinated PRINT particles (41.67 4) generated in Example 1 were incubated with NRPV7-Kd (3.28 μL of a 2.08 μg/μL solution) or HA-Kd (2.44 μL of a 2.88 μg/μL solution) on ice for 30 min followed by quenching of remaining biotin binding sites with 5 μl of 500 μM biotin solution. The particles were incubated on ice for an additional 10 min.

MHC Preparation Bacteria were transformed with a MHC I plasmid coding for the D^{b} MHC I molecules with a 15 amino acid tag (GLNDIFEAQKIEWHE). The tag conferred the ability to biotinylate the protein with the enzyme BirA. The bacteria were grown in 13 liters of selective medium. The bacteria were pelleted and passed through a French Press at 16,000 psi, and the MHC molecules isolated from the inclusion bodies in 8M urea. The MHC I molecules were refolded with the peptide of interest in the presence of beta-2-microglobulin for 24-36 hours at 10 degrees C in 1 liter of Refolding buffer (Refolding buffer=100 mM Tris (pH=8.0), 400 mM L-Arginine, 2 nM EDTA, 1 ug/ml leupeptin, 2 ug/ml aprotinin, 4.9 nM GSH (glutathione reduced), 0.49 mM GSSH (glutathione oxidized). The refolded MHC/peptide/beta2M complexes were concentrated to 25 ml in a nitrogen pressure filtration device with a 10,000 MWCO filter and then concentrated to 1 ml in a Centricon with a 10,000 MWCO filter. The MHC/peptide/beta2M was then purified through a HPLC column.

The spleens from one NOD-CL4 mouse and one NOD-8.3 mouse were isolated and disassociated. The cells were resuspended in Ammonium chloride Red Blood Cell Lysis Buffer (0.15 M NH₄Cl, 1. M KHCO₃, 0.1 mM Na₂EDTA, pH to 7.2-7.4), washed 2 times, and strained (Falcon # 352340) and counted. The spleenocytes were blocked in FcR Block (24G2, B-cell hybridoma supernatant) for 20 minutes on ice, at a concentration of 1×10⁶ cells per 10 μL of FcBlock. Antibodies were added in an additional volume of 40 μL, and PRINT particles were added in an additional 50 μL, for a total volume of 100 μL (dilutions were done in FACS WASH (=2% FBS, 0.1% NaN₃, PBS)). The antibodies and PRINT particles were incubated with the spleenocytes on ice and in the dark for an additional 25 minutes, and then washed 3 times in FACS WASH prior to analysis on the Cyan FACS machine.

In the 8.3 NOD mouse, 75% of their CD8+Tcells are transgenic and will recognize the NRP-V7 MHCl (Kd) tetramer. In the CL4-NOD mice, almost all of their CD8+Tcells are transgenic and will recognize the HA-MHCl (Kd) tetramer. The NRP-V7-Kd coated PRINT particles were found to target around 75% of the CD8+Tcells in the spleen of the 8.3-NOD mice but only 4% of the CD8+Tcells in the spleen of the CL4-NOD mice. The HA-Kd coated PRINT particles targeted 94% of the CD8+Tcells in the spleen of the HA-NOD mice but only 1% of the CD8+Tcells in the spleen of the 8.3-NOD mice.

### Example 37

### In Vitro Studies of Disulfide-Based Rectangular Particles with Antibody-Functionalized Surfaces Containing Ova Peptide

A patterned FLUOROCUR™ based mold was generated by pouring FLUOROCUR material over a silicon substrate patterned with 2×2×1 μm rectangles. The FLUOROCUR material covered master was then subjected to UV light (λ=365 nm) for 3 minutes while under a nitrogen purge. The fully cured FLUOROCUR based mold was then released from the silicon master. Separately, a mixture of 57 mg trimethylolpropane ethoxylate triacrylate (˜900 MW), 20 mg cystaminebisacrylamide or PEG₂₀₀-diacrylate (for a non-degradable particle), 10 mg 2-amino-ethyl-methacrylate, 2 mg ova peptide (SIINFEKL), 1 mg 1-hydroxycyclohexylphenyl ketone was prepared in 50 μL DMSO. This mixture was spotted directly onto the patterned Fluorocur based mold and covered with an unpatterned raw PET film. The monomer mixture was pressed between the two polymer sheets, and spread using a roller. The mold and PET sheet were then passed through a heated laminator (10 V heating, 7 V rolling). The mold was delaminated as it came out of the laminator. The mold was then subjected to UV light (λ=365 nm) for 2 minutes while maintaining a nitrogen purge. The particles were mechanically harvested by placing 5 mL of water on the mold and scraping the surface with a glass slide. The particle suspension was transferred to a scintillation vial. PBS (0.5 mL, 10×) was added followed by Sulfo-NHS-LC-Biotin (200 μL, 9.4 mg/mL in DMSO) and the mixture was stirred for 1 h. Particles were collected from solution onto a centrifugal filter membrane (0.65 μm pore size, PVDF membrane, Millipore) and then washed with 3 mL of water to remove any excess NHS-LC-biotin. The particles were resuspended in 2 mL of water.

Streptavidin AlexaFluor 647 (200 μL, 2 mg/mL in PBS, Invitrogen) was added to the particle solution above. After stirring for 1 h, particles were again collected onto a centrifugal filter membrane (0.65 μm pore size, PVDF membrane, Millipore) and then washed with 1 mL of water to remove any unbound streptavidin. The particles were resuspended in 2 mL of water.

Anti-mouse CD11b (100 μL, 0.5 mg/mL in PBS, eBiosciences) was added to the particle solution from above. The mixture was stirred for 1 h. Particles were collected onto a centrifugal filter membrane (0.65 μm pore size, PVDF membrane, Millipore) and washed with 1 mL of water to remove any unbound antibody. The particles were resuspended in 0.4 mL of water and PBS (45 μL, 10×) was added.

In vitro delivery: 44.4 ul of sterile filtered 0.5M reduced-glutathione (Sigma G6529)(Mw=307.32 g/mole) in PBS was added to the 200 μl of PRINT particles to make a final concentration of 100 mM solution of glutathione on the PRINT particles. The particles were incubated overnight at 4 C.

The spleen from one male C57B/6 four week old mouse was isolated and disassociated with two tweezers. The large particles were allowed to settle and the cells were transferred to a conical tube. The cell were centrifuged at 1900 rpm at 4 degrees C. for 5 minutes and resuspended in Ammonium chloride Red Blood Cell Lysis Buffer (ACh buffer, 0.15 M NH₄Cl, 1. M KHCO₃, 0.1 mM Na₂EDTA, pH to 7.2-7.4) on ice for 2 minutes then washed 2 times and strained through a cell strainer (Falcon # 352340) to remove the clumps. The cells were counted. 1.2×10⁸ cells were obtained from 1 B6 mouse spleen. The cells were resuspended in RPMI/Na Pyruvate/pen/strep/glut/55 microM b-ME/10% FBS.

The PRINT+glutathione digestion was centrifuged at max speed at 4 C for 30 minutes; the 244 ul of supernatant was transferred to separate eppendorf tubes and 244 ul of RPMI was added to the PRINT particle pellet. Then, 3×10⁵ spleenocytes were plated per well in two 96 well plates in RPMI, 10% FBS, L-glutamine, Na Pyruvate, pen/strep, and 55 uM b-mercaptoethanol at 100 ul/well. 50 ul/well of peptide, glutathione treatment supernatant, or PRINT particle solutions were added to each well and the plates were incubated at 37 C overnight (20 hours). Then, 3×10⁴ B3Z cells/well (50 μL) were added for a total of 200 ul/well. The plates were incubated at 37 C for 20 hours. The plates were removed and spun at 1600 rpm for 5 minutes to pellet the cells, and the medium was gently discard. The cells were washed with 100 μl of PBS, and the 96 well plate was spun at 1600 rpm for 5 minutes. The PBS was discarded. Then, CPRG (100 μl/well of 91 μg/ml) in Z-Buffer (100. mM beta-mercaptoethanol, 9. mM MgCl₂, 0.125% NP40, in PBS) was added and the plates incubated for 4 h. STOP Buffer (100 μl/well, 300 mM glycine, 15 mM EDTA, in water) was added, and the OD at 570 and 650 was measured using an ELISA Plate Reader (Molecular Devices SpectraMax-M2, Software=SoftMaxPro v5).

The absorbance at 570 nm is directly proportional to T cell activation. (Kwon, Y. J.; James, E.; Shastri, N.; Frechet, J. M. J., In vivo targeting of dendritic cells for activation of cellular immunity using vaccine carriers based on pH-responsive microparticles. Proceedings of the National Academy of Sciences of the United States of America 2005, 102, (51), 18264-18268.) It is due to the production of Lac-Z by T cells, which can only be stimulated by the proper presentation of ova peptide-MHC complexes by dendritic cells. Degradable disulfide-based PRINT particles were much more efficient than non-degradable diacrylate-based PRINT particles at stimulating T cells as measured by the absorbance at 570 nm implying that ova release was due to particle degradation and not passive diffusion. Results for cells dosed with PRINT particles, supernatant from glutathione treatment, and free peptide are shown in FIG. 32.

1. A drug delivery vehicle, comprising:
a substantially predetermined shape and a volume less than about 150 μm³;
a crosslinked matrix; and
a biologically active cargo;
wherein the crosslinked matrix is configured to controllably biodegrade to release the biologically active cargo from the vehicle.

2. The drug delivery vehicle of claim 1, wherein the biologically active cargo comprises an oligonucleotide.

3. The vehicle of claim 3, wherein the crosslinked matrix includes a biodegradable crosslinker.

4. The vehicle of claim 1, wherein the biodegradable crosslinker comprises a disulfide.

5. The vehicle of claim 1, wherein a concentration of the cargo associated with the vehicle is not in an equilibrium state.

6. The vehicle of claim 1, wherein the cargo comprises less than about 75 weight percent of the particle.

7. The vehicle of claim 2, wherein the oligonucleotide comprises RNA, siRNA, shRNA, dsRNA, ssRNA, miRNA, rRNA, tRNA, snRNA, DNA, ssDNA, dsDNA, plasmid DNA, antisense RNA, or vaccine.

8. The vehicle of claim 2, wherein the volume of the vehicle is not dependent on a parameter selected from the group consisting of size of the oligonucleotide, a concentration of the oligonucleotide, a charge of the oligonucleotide, charge density of the oligonucleotide, or chain length of the oligonucleotide.

9. The vehicle of claim 1, wherein the crosslinked matrix comprises poly(ethylene glycol).

10. The vehicle of claim 1, wherein the crosslinked matrix comprises a crosslinker that is biodegradable in a predetermined environment selected from the group of an intracellular environment, a preselected pH, an enzyme, temperature, radiation, magnetic field, a reducing environment, and an oxidative environment.

11. A drug delivery vehicle, comprising:
a substantially predetermined shape and a volume less than about 150 μm³;
a matrix; and
an oligonucleotide;
wherein the matrix is configured to control diffusion of the oligonucleotide from the vehicle.

12. The vehicle of claim 11, wherein the matrix includes a crosslinked polymer.

13. The vehicle of claim 12, wherein the crosslinked polymer comprises a hydrogel.

14. The vehicle of claim 11, wherein the oligonucleotide passively diffuses from the matrix.

15. The vehicle of claim 11, wherein a concentration of the oligonucleotide associated with the vehicle is not in an equilibrium state.

16. The vehicle of claim 11, wherein the oligonucleotide comprises RNA, siRNA, shRNA, dsRNA, ssRNA, miRNA, rRNA, tRNA, snRNA, DNA, ssDNA, dsDNA, plasmid DNA, antisense DNA, antisense RNA, or vaccine.

17. The vehicle of claim 11, wherein the volume of the vehicle is not dependent on a parameter selected from the group consisting of size of the oligonucleotide, a concentration of the oligonucleotide, a charge of the oligonucleotide, charge density of the oligonucleotide, or chain length of the oligonucleotide.

18. The vehicle of claim 11, wherein the matrix comprises poly(ethylene glycol).

19. The vehicle of claim 11, wherein the matrix comprises a crosslinker that is biodegradable in a predetermined environment selected from the group of an intracellular environment, a preselected pH, an enzyme, a reducing environment, and an oxidative environment.

20. A method for fabricating a drug delivery vehicle, comprising:
introducing a composition comprising a biologically active cargo and a crosslinkable matrix into a cavity of a mold, wherein the mold is fabricated from a non-wetting polymer and wherein the cavity has predetermined shape and a volume of less than about 150 μm³;
forming a particle from the composition in the cavity, wherein the particle includes a crosslinked matrix with a crosslink density; and
extracting the particle from the cavity to yield an isolated particle having a substantially predetermined shape and a volume less than about 150 μm³ and a composition comprising a crosslinked matrix and a biologically active cargo.

21. The method of claim 20, wherein the biologically active cargo comprises an oligonucleotide.

22. The method of claim 20, wherein the crosslinked matrix comprises a biodegradable crosslinker.

23. The method of claim 22, wherein the biodegradable crosslinker comprises disulfide.

24. The method of claim 20, wherein the particle is configured to controllably biodegrade based on the crosslink density.

25. The method of claim 20, wherein the particle comprises a hydrogel.

26. A method for releasing a drug from a drug delivery vehicle, comprising:
introducing a composition comprising an oligonucleotide into a cavity of a mold, wherein the cavity is fabricated from a non-wetting polymer and wherein the cavity has a predetermined shape and a volume of less than about 150 μm³;
forming a particle from the composition in the cavity;
extracting the particle from the cavity to yield an isolated particle having a volume less than about 150 μm³ and a biodegradable composition comprising the oligonucleotide; and
actively or passively releasing the oligonucleotide from the particle.

27. The method of claim 26, wherein the oligonucleotide is passively released without breaking chemical bonds of the particle.

28. The method of claim 26, wherein the oligonucleotide is passively released by swelling of the particle, diffusion of the oligonucleotide from the particle, pore size of the particle, oligonucleotide volume in relation to particle volume, or affinity of the oligonucleotide with the particle.

29. The method of claim 26, wherein the oligonucleotide is actively released by breakage of chemical bonds of the particle.

30. A method of treating a disease with a therapeutic agent, comprising:
introducing a composition comprising a crosslinkable matrix and a therapeutic agent into a cavity of a mold, wherein the mold is fabricated from a non-wetting polymer and wherein the cavity has a predetermined shape and a volume of less than about 150 μm³;
forming a particle from the composition in the cavity, wherein the particle is configured to actively or passively release the therapeutic agent;
extracting the particle from the cavity to yield an isolated particle having a substantially predetermined shape, a volume less than about 150 μm³ and a biodegradable composition comprising the therapeutic agent; and
delivering the particle to a patient wherein the particle crosses a cellular membrane into intracellular space and actively or passively releases the therapeutic agent.

31. The method of claim 30, wherein the therapeutic agent is passively released without breaking chemical bonds of the particle.

32. The method of claim 30, wherein the therapeutic agent is passively released by swelling of the particle, diffusion of the therapeutic agent from the particle, pore size of the particle, therapeutic agent volume in relation to particle volume, or affinity of the therapeutic agent with the particle.

33. The method of claim 30, wherein the therapeutic agent is actively released by breakage of chemical bonds of the particle.

34. A composition comprising:
a plurality of vehicles wherein each vehicle of the plurality of vehicles comprises;
a substantially predetermined shape;
a volume less than about 150 μm³;
a crosslinked matrix; and
an oligonucleotide;
wherein the crosslinked matrix is configured to controllably biodegrade to release the oligonucleotide from the vehicle; and
wherein each substantially predetermined shape is substantially equivalent.

35. The vehicle of claim 34, wherein the crosslinked matrix includes a biodegradable crosslinker.

36. The vehicle of claim 35, wherein the biodegradable crosslinker comprises a disulfide.

37. A method of diagnosing a disease with a diagnostic agent, comprising:
introducing a composition comprising a crosslinked matrix and a diagnostic agent into a cavity of a mold, wherein the mold is fabricated from a non-wetting polymer and wherein the cavity has a predetermined shape and a volume of less than about 150 μm³;
forming a particle from the composition in the cavity, wherein the particle is configured to release the diagnostic agent from the particle;
extracting the particle from the cavity to yield an isolated particle having a substantially predetermined shape, a volume less than about 150 μm³ and a biodegradable composition comprising a diagnostic agent; and
delivering the particle to a patient wherein the particle crosses a cellular membrane into intracellular space and releases the diagnostic agent.

38. The method of claim 37, wherein the diagnostic agent is passively released without breaking chemical bonds of the particle.

39. The method of claim 37, wherein the diagnostic agent is passively released by swelling of the particle, diffusion of the diagnostic agent from the particle, pore size of the particle, diagnostic agent volume in relation to particle volume, or affinity of the diagnostic agent with the particle.

40. The method of claim 37, wherein the diagnostic agent is actively released by breakage of chemical bonds of the particle.

41. A functionalized delivery vehicle, comprising:
a matrix comprising;
a crosslinker;
a biologically active cargo; and
a functional amine group;
wherein the matrix is configured and dimensioned into a substantially predetermined shape and a volume less than about 150 cubic micrometers; and
wherein the functional amine group is coupled with an antigen.

42. The functionalized delivery vehicle of claim 41, wherein the antigen comprises an MHC-antigen.
